(19) United States
(12) Patent Application Publication
(10) Pub. No.: US 2024/0306650 A1
(43) Pub. Date: Sep. 19, 2024

(54) MICROALGAE COMPOSITIONS FOR HOST PLANT NUTRIENT UTILIZATION, ABIOTIC STRESS, AND SOIL FITNESS

(71) Applicant: AE Agribiologicals S.L., Madrid (ES)

(72) Inventors: Xing Liang LIU, Davis, CA (US); Douglas Ry WAGNER, Pleasant Hill, OR (US); Miguel Garcia GUERRERO, Seville (ES)

(21) Appl. No.: 18/673,144

(22) Filed: May 23, 2024

Related U.S. Application Data
(63) Continuation of application No. PCT/EP2022/083062, filed on Nov. 23, 2022.
(60) Provisional application No. 63/347,818, filed on Jun. 1, 2022.
(60) Provisional application No. 63/283,182, filed on Nov. 24, 2021.

(51) Int. Cl. A01N 65/03; A01N 25/12; A01P 21/00

(52) U.S. Cl. CPC A01N 65/03; A01N 25/12; A01P 21/00

(57) ABSTRACT

The present disclosure provides methods for improving the nutrient utilization, resistance to abiotic stress, and recovery from abiotic stress of a host plant by applying a microalgae composition to the host plant. The disclosure also provides methods for improving soil fitness and improving rhizospheric activity and/or growth by applying a microalgae composition to the soil.

### CROSS-REFERENCE TO RELATED APPLICATIONS

This application claims the benefit of and priority to U.S. Provisional Patent Application No. 63/283,182, filed on Nov. 24, 2021, and U.S. Provisional Patent Application No. 63/347,818, filed on Jun. 1, 2022, the contents of each of which are herein incorporated by reference in their entireties.

### FIELD OF THE DISCLOSURE

The present disclosure relates to methods for improving host plant nutrient utilization, methods for improving host plant response to abiotic stress, e.g., water stress, and methods for improving soil fitness via application of compositions comprising microalgae-derived components.

### BACKGROUND

Modern agricultural practices involve the use of high-yielding, disease-resistant crop varieties, and the constant input of agrochemicals such as chemical fertilizers and pesticides. The application of such chemicals can adversely affect the dynamic equilibrium of the soil, detriment the environment, and decrease agricultural biodiversity by destroying useful microorganisms that provide critical nutrition and active natural compounds to promote crop growth and development.

In conjunction with farming practices, there is a growing need for new methods of improving host plant nutrient utilization and soil fitness in an ecologically safe and sustainable way.

### BRIEF SUMMARY

In one aspect, the present disclosure provides a method for improving host plant nutrient utilization, the method comprising the step of: a) applying a microalgae-based formulation to the host plant.

In some embodiments, the method upregulates expression of genes involved in nitrogen metabolism and/or amino acid metabolism. In some embodiments, the method decreases the amount of exogenous nitrogen required by the host plant. In some embodiments, the method decreases the amount of exogenous nitrogen required by the host plant by at least 25% without negatively impacting host plant yield. In some embodiments, the method produces the same or higher value of a growing parameter, production parameter, or biostimulant parameter compared to a control plant without the microalgae-based formulation, with less exogenous nitrogen application than the control plant.

In one aspect, the present disclosure provides a method for improving soil fitness, the method comprising the step of: a) applying a microalgae-based formulation to the soil.

In some embodiments, the method enhances nutrient availability in the soil. In some embodiments, the method increases the soil content of a nutrient selected from the list consisting of: phosphate, assimilable phosphorous, organic carbon, water soluble carbon, humic substances, total nitrogen, water soluble nitrogen, nitrate, and water soluble polyphenols. In some embodiments, the method changes the nutrient composition of the soil, but does not change the nutrient composition of the leaves of a host plant planted therein.

In one aspect, the present disclosure provides a method for stimulating rhizospheric growth and activity within a soil, the method comprising the step of: a) applying a microalgae-based formulation to the soil.

In some embodiments, the method stimulates microbial development and/or respiration. In some embodiments, the method increases the level of alkaline phosphatase activity, beta-glucosidase activity, urease activity, basal respiration, glycine aminopeptidase activity, and/or hydrolase activity in the soil. In some embodiments, the method increases the biodiversity of the soil microbiome.

In one aspect, the disclosure provides a method for improving host plant resistance to and/or recovery from abiotic stress, the method comprising the step of: applying a microalgae-based formulation to the host plant. In some embodiments, the abiotic stress is water stress, temperature stress, sun stress, salinity stress, wind stress, herbicidal stress, or heavy metal stress. In some embodiments, the abiotic stress is drought, heat, cold, excess salinity, herbicide exposure, strong winds, heavy metals, flooding, or excessive sunlight. In some embodiments, the abiotic stress is drought.

In some embodiments, the method decreases the damage to a growing parameter, production parameter, or biostimulant parameter of the host plant incurred by exposure to the abiotic stress. In some embodiments, the method decreases loss of yield as a result of exposure to the abiotic stress. In some embodiments, the method improves recovery from the damage to a growing parameter, production parameter, or biostimulant parameter of the host plant incurred by exposure to the abiotic stress.

In some embodiments, the method upregulates expression of genes involved in resistance to abiotic stress and/or drought resistance. In some embodiments, the method upregulates expression of a gene encoding an ABA receptor, a protein phosphatase 2C, an SNF1-related protein kinase 2, an ABRE-binding protein, an ABRE-binding factor, and/or a mitogen-activated protein kinase.

In some embodiments, the formulation comprises multiple species of microalgae.

In some embodiments, the formulation comprises microalgae from a phylum selected from the list consisting of: Chlorophyta, Cryptophyta, Cyanophyta, Euglenophyta, Heterokontophyta, or Rhodophyta.

In some embodiments, the formulation comprises microalgae from a genus selected from the list consisting of: Chlorella, Scenedesmus, Nannochloropsis, Muriellopsis, Isochrysis, Tisochrysis, Desmodesmus, Haematococcus, Arthrospira, and Anabaena.

In some embodiments, the formulation comprises whole-cell microalgae powder.

In some embodiments, the formulation comprises 0.1-50 g/L of whole-cell microalgae powder. In some embodiments, the formulation comprises 0.8-20 g/L of whole-cell microalgae powder. In some embodiments, the formulation comprises digested microalgae solution (“DMS”). In some embodiments, the formulation comprises 0.3-0.5% v/v DMS. In some embodiments, the formulation comprises 5-15% dry matter of microalgae. In some embodiments, the formulation comprises 5-15% dry matter of microalgae and is diluted to 0.3-0.5% v/v in water prior to application. In some embodiments, the formulation is a liquid, and the formulation is applied at a rate of 0.5-20 L/ha. In some embodiments, the formulation is a liquid, and the formulation is applied at a rate of 1-10 L/ha. In some embodiments, the formulation is a granule formulation, and the formulation is applied at a rate of 1-20 kg/ha. In some embodiments, the formulation is a granule formulation, and the formulation is applied at a rate of 5-15 kg/ha.

### BRIEF DESCRIPTION OF THE SEVERAL VIEWS OF THE DRAWINGS

FIG. 1 shows a nutrient analysis of an illustrative digested microalgae solution (“DMS”) of the disclosure.

FIG. 2A shows tomato yield results in average total pounds per plot of application in each treatment condition. FIG. 2B shows number of large, medium, and small tomatoes between DMS treated and untreated conditions with 75% nitrogen.

FIG. 3A shows examples of groups of genes upregulated in juvenile and mature tomato plants at 2 hr time points. 24 hour time points, or both time points. FIG. 3B shows examples of gene categories and number of identified genes in different treatment groups. FIG. 3C shows examples of genes related to amino acid and nitrogen metabolism upregulated in juvenile tomato plants at 2 hours. FIG. 3D shows examples of genes related to amino acid and nitrogen metabolism upregulated in juvenile tomato plants at 24 hours and mature tomato plants at 2 hours. FIG. 3E shows an overview of a biosynthesis pathway highlighting upregulated genes involved in nitrogen and amino acid metabolism. FIG. 3F shows a table of upregulated genes involved in amino acid metabolism. FIG. 3G shows images demonstrating improved drought resistance in plants treated with an illustrative DMS of the disclosure.

FIG. 4A-40 show the effects of DMS treatment on soil composition for young almond trees. The figures show the changes to organic C (FIG. 4A): water soluble C (FIG. 4B): water soluble polyphenols (FIG. 4C); humic substances (FIG. 4D); total N (FIG. 4E); water soluble N (FIG. 4F); nitrate (FIG. 4G): assimilable P (FIG. 4H): alkaline phosphatase activity (FIG. 4I): beta-glucosidase activity (FIG. 4J); urease activity (FIG. 4K): basal respiration (FIG. 4L): structural microbiome biodiversity in terms of bacteria and total PLFAs (FIG. 4M): structural microbiome biodiversity in terms of gram positive bacteria, gram negative bacteria, and fungi (FIG. 4N); and functional biodiversity in terms of use of carbon sources (FIG. 4O).

FIG. 5A-5K show the effects of DMS treatment on soil composition for old almond trees. The figures show the changes to organic C (FIG. 5A); water soluble C (FIG. 5B): humic substances (FIG. 5C): total N (FIG. 5D); assimilable P (FIG. 5E): alkaline phosphatase activity (FIG. 5F): glycine aminopeptidase activity (FIG. 5G): beta glucosidase activity (FIG. 5H): basal respiration (FIG. 5I): structural microbiome biodiversity in terms of bacteria and total PLFAs (FIG. 5J); and functional biodiversity in terms of use of carbon sources (FIG. 5K).

FIG. 6 shows a table demonstrating the effect of DMS treatment on the soil of lettuce leaves in terms of changes to phosphate and assimilable P content.

FIG. 7A-7D show improved resistance to drought stress in a DMS-treated tomato plant (left) versus in a control untreated tomato plant (right) on day 3 (FIG. 7A), day 6 (FIG. 7B), day 13 (FIG. 7C), and day 16 (FIG. 7D) in drought conditions.

FIG. 8A-8E show improved recovery from drought stress in a DMS-treated tomato plant (left) versus in a control untreated tomato plant (right) on day 17 before regular watering (FIG. 8A), day 17 after regular watering (FIG. 8B), day 18 (FIG. 8C), day 20 (FIG. 8D), and day 28 (FIG. 8E) in drought recovery conditions.

FIG. 9A-9E show the effect of DMS foliar treatment in reducing the impact of herbicidal stress in eggplant crops due to herbicidal drift from neighboring fields. FIG. 9A shows neighboring untreated control plants and DMS treated plants exhibiting significant visual differences in growth and coloring in response to herbicidal stress. FIG. 9B shows healthy height, width, number of leaves and color of leaves in DMS-treated plants exposed to herbicidal drift. FIG. 9C shows stunted growth, decreased leafing, curled leaves, and browning leaves in untreated control plants. FIG. 9D shows close up images of plants in the DMS treatment group, demonstrating good plant health, coloring, and flowering with little to no evidence of herbicidal stress. FIG. 9E shows close up images of plants from the untreated control group demonstrating curling, wilting, yellowing, and dying leaves, as well as dropped flowers.

FIG. 10A shows some of the genes involved in the host plant response to abiotic stress, e.g., drought stress. FIG. 10B shows some of the genes upregulated at various stages related to abiotic stress, e.g., drought stress.

FIG. 11A shows analysis of microbial populations as a function of sampling date, while FIG. 11B shows analysis of microbial populations as a function of treatment condition. Permanova analysis in both cases yielded p<0.01.

FIG. 12A-12E show nutrient analysis in treated soil compared to control soil at time points T1 and T2. FIG. 12A shows results of carbon nutrient analysis: FIG. 12B, nitrogen nutrient analysis: FIG. 12C, phosphorous nutrient analysis: FIG. 12D, potassium nutrient analysis; and FIG. 12E, minor nutrient analysis. * indicates 0.1<p<0.3.

FIG. 13 shows the analysis of stress adaptation pathways in treated soil compared to control soil at time points T1 and T2. Dashed line around the result indicates change observed in multiple samples and/or time points, p>0.3.

FIG. 14 shows the analysis of biocontrol pathways, in treated soil compared to control soil at time points T1 and T2. Dashed line around the result indicates change observed in multiple samples and/or time points, p>0.3.

FIG. 15A-15D show the results of a corn field trial testing reduced NPK reliance with DMS treatment. FIG. 15A shows average corn yield (kg/ha): FIG. 15B shows average humidity (%): FIG. 15C shows fertilizer costs (€/ha); and FIG. 15D shows projected return on investment (€/ha).

FIG. 16A-16E show the results of a barley field trial testing reduced N reliance with DMS treatment. FIG. 16A shows average barley yield (kg/ha): FIG. 16B shows average mass per hectoliter (kg/hl): FIG. 16C shows percent protein: FIG. 16D shows projected return on investment (€/ha); and FIG. 16E shows NDVI results across treatment conditions at three time points.

### DETAILED DESCRIPTION

### Definitions

The term “a” or “an” refers to one or more of that entity, i.e. can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 15% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

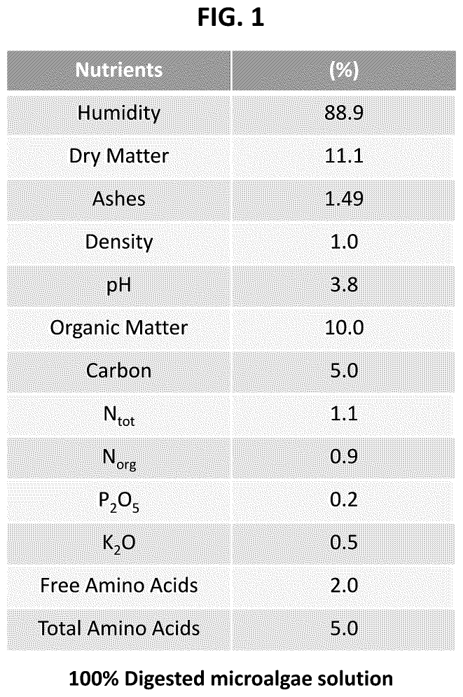

As used herein, “microalgae” are eukaryotic microbial organisms that contain a chloroplast or other plastid, and optionally, are capable of performing photosynthesis and prokaryotic microbial organisms capable of performing photosynthesis. Microalgae include obligate photoautotrophs, which are organisms that use light energy (e.g. from sunlight or other light source) to convert inorganic materials into organic materials for use in cellular functions such as biosynthesis and respiration. Microalgae also include heterotrophs, which can live solely off of a fixed carbon source. Microalgae include unicellular organisms that separate from sister cells shortly after cell division, as well as microbes such as, for example, Volvox, which is a simple multicellular photosynthetic microbe of two distinct cell types. Microalgae also include other microbial photosynthetic organisms that exhibit cell-cell adhesion, such as Agmenellum, Anabaena, and Pyrobotrys. In some embodiments, the microalgae of the present disclosure are selected from the phyla Chlorophyta, Cryptophyta, Cyanophyta, Euglenophyta. Heterokontophyta, and Rhodophyta. In some embodiments, the microalgae of the present disclosure are selected from the genera Chlorella, Scenedesmus, Nannochloropsis, Muriellopsis, Isochrysis, Tisochrysis, Desmodesmus, Haematococcus, Arthrospira, and Anabaena. As used in this description, the term microalgae encompasses any form of microalgae, whether in a natural and unprocessed whole state, dried, extracted, or otherwise processed. In some embodiments, the term “microalgae” is used to refer to a lysed, hydrolyzed, digested, pulverized, or otherwise processed form of microalgae. In some embodiments, microalgae used in the compositions herein has the nutrient analysis depicted in FIG. 1. In some embodiments, microalgae is not macroalgae. In some embodiments, microalgae as used in the present compositions is not live microalgae.

As used herein, a “composition comprising microalgae” or “microalgae composition” refers to a composition comprising microalgae-derived components. Compositions comprising microalgae according to the present disclosure comprise, e.g., dried whole cell microalgae and/or lysed and digested microalgae. “Whole cell microalgae powder” refers to microalgae that has been dried and ground after being harvested. “Digested microalgae solution” or “DMS” refers to microalgae that has been dried, ground, and then processed to degrade cell walls and release peptides and other nutrients. DMS can be formulated using chemical, physical, or biological means to degrade cell walls and release peptides. As used herein, “microalgae dry matter” or “dry matter of microalgae” refers to the non-liquid content of a composition comprising microalgae.

As used herein, the terms “mycorrhiza” and “mycorrhizae” refer to mycorrhizal fungi. A mycorrhiza is a mutual symbiotic association between a fungus and a plant and the term is also used herein to refer to the fungus itself. “Ectomycorrhizae” is used to refer to mycorrhizal fungi that colonize host plant root tissues extracellularly. “Endomycorrhizae” is used to refer to mycorrhizal fungi that colonize host plant tissues intracellularly. In some embodiments, the compositions of the present disclosure comprise both ectomycorrhizae and endomycorrhizae. In some embodiments, the compositions of the present disclosure comprise predominantly endomycorrhizae, e.g., more than 90% endomycorrhizae.

As used herein, a “granule” refers to a dry, granular composition having an average diameter of less than about 1 cm for administration to agricultural crops.

As used herein, a “seed coating” refers to a composition applied to the seeds of an agricultural crop before or during planting.

As used herein, an “agricultural crop” refers to any plant that is harvested for commercial purposes. Agricultural crops include agronomic crops, horticultural crops, and ornamental plants. “Agronomic crops” are those that occupy large acreage and are the bases of the world's food and fiber production systems, often mechanized. Examples are wheat, rice, corn, soy bean, alfalfa and forage crops, beans, sugar beets, canola, and cotton. “Horticultural crops” are used to diversify human diets and enhance the living environment. Vegetables, fruits, flowers, ornamentals, and lawn grasses are examples of horticultural crops and are typically produced on a smaller scale with more intensive management than agronomic crops. “Ornamental plants” are grown for decoration and include flowers, shrubs, grasses, and trees. Agricultural crops include both monocots and dicots. Monocots include most of the bulbing plants and grains, including agapanthus, asparagus, bamboo, bananas, corn, daffodils, garlic, ginger, grass, lilies, onions, orchids, rice, sugarcane, tulips, and wheat. Dicots include many garden flowers and vegetables, including legumes, the cabbage family, and the aster family. Examples of dicots are apples, beans, broccoli, carrots, cauliflower, cosmos, daisies, peaches, peppers, potatoes, roses, sweet pea, and tomatoes. Agricultural crops also include food crops, feed crops, cereal crops, oil seed crop, pulses, fiber crops, sugar crops, forage crops, medicinal crops, root crops, tuber crops, vegetable crops, fruit crops, and garden crops. The terms “host plant” and “agricultural crop” are used interchangeably herein.

As used herein, the term “carrier” is intended to include an “agronomically acceptable carrier.” An “agronomically acceptable carrier” is intended to refer to any material which can be used to deliver a composition as described herein, alone or in combination with one or more agriculturally beneficial ingredient(s), and/or biologically active ingredient(s), to a plant, a plant part (e.g., a leaf or a seed), or a soil. In some embodiments, the carrier can be added to the plant, plant part or soil without having an adverse effect on plant growth or soil fitness.

As used herein, “soil fitness” broadly refers to parameters of soil health including nutrient concentration, water soluble nutrient concentration, host plant available nutrient concentration, nutrient cycling, microbial concentration, microbial diversity, microbial respiration, and microbial activity.

As used herein, “nutrient utilization” refers to a host plant's ability to utilize available nutrients. In some embodiments, the methods herein improve a host plant's ability to utilize available nutrients. In some embodiments, the nutrient is a macronutrient or a micronutrient. In some embodiments, the nutrient is nitrogen. In some embodiments, the methods herein improve a host plant's ability to utilize available nitrogen thereby decreasing reliance on exogenous nitrogen.

As used herein, a “growing parameter” of a host plant is related to the growth of the host plant. Growing parameters include plant size, biomass (dry or wet), aerial biomass, height, number of branches, number of leaves, number of flowers, root biomass, number of roots, number of secondary roots, root volume, root length, and degree of inoculation by diazotrophic bacteria.

As used herein, a “production parameter” of a host plant is related to the plant part that is harvested from the plant for commercial purposes. Production parameters include, but are not limited to, yield, yield per plant, yield per area, harvested biomass, harvested weight, harvested volume, number of harvested plant parts, and size of harvested plant parts. In terms of the harvestable plant parts, production parameters include yield, weight, size, and number of harvestable plant parts. Harvestable plant parts include, for example, fruits, vegetables, roots, grains, tubers, leaves, flowers, seeds, and nuts. In some embodiments, e.g., for some grasses, lettuces, feed crops, and forage crops, a harvestable plant part is the entire aerial biomass of the plant. In some embodiments, the harvestable plant part is related to the intended use of the crop. For example, for oil crops, the harvestable plant parts are the components of the plant containing the oil to be harvested.

As used herein, a “biostimulant parameter” of a host plant refers to a parameter having to do with the molecular makeup of the host plant. Biostimulant parameters include, but are not limited to, chlorophyl content, carotenoid content, micronutrient profile, and macronutrient profile. In some embodiments, the method increases the concentration of a chlorophyl, e.g., chlorophyl a or chlorophyl b.

### Compositions Comprising Microalgae

The present disclosure relates to compositions comprising microalgae. In some embodiments, the compositions comprise dried whole cell or digested microalgae.

### Microalgae

Within the present compositions, microalgae are eukaryotic microbial organisms that contain a chloroplast or other plastid, and optionally, are capable of performing photosynthesis, and prokaryotic microbial organisms capable of performing photosynthesis. Microalgae may exist individually, or in chains or groups and can range in size from a few micrometers to a few hundred micrometers. Microalgae do not have roots, stems, or leaves. Microalgae capable of performing photosynthesis are important for life on earth: they produce approximately half of the atmospheric oxygen and use simultaneously the greenhouse gas carbon dioxide to grow photoautotrophically. Microalgae, together with bacteria, form the base of the food web and provide energy for all the trophic levels above them. Microalgae biomass is often measured with chlorophyll a concentrations and can provide a useful index of potential production. Microalgae include obligate photoautotrophs, which cannot metabolize a fixed carbon source as energy, as well as heterotrophs, which can live solely off of a fixed carbon source.

The compositions of the present disclosure comprise microalgae. In some embodiments, the compositions comprise microalgae of a phylum selected from the list consisting of: Cyanobacteria, Chlorophyta, Rhodophyta, Bacillariophyta, Cryptophyta, Dinophyta, Euglenozoa, Haptophyta, Ochrophyta, Cyanophyta, Euglenophyta, Heterokontophyta, and Rhodophyta. In some embodiments, the microalgae included in compositions of the present disclosure are selected from the phyla Chlorophyta, Cryptophyta, Cyanophyta, Euglenophyta, Heterokontophyta, and Rhodophyta.

In some embodiments, the microalgae are of a genus selected from the list consisting of: Anabaena, Aphanizomenon, Arthrospira, Auxenochlorella, Botryococcus, Carteria, Chaetoceros, Chlamydomonas, Chlorella, Chlorococcum, Chroomonas, Coccomyxa, Crypthecodinium, Cryptomonas, Cyclotella, Desmodesmus, Dicrateria, Dunaliella, Euglena, Haematococcus, Isochrysis, Microcystis, Micromonas, Monochrysis, Muriellopsis, Nannochloropsis, Navicula, Neochloris, Nitzschia, Nostoc, Olisthodiscus, Phaeodactylum, Pseudoisochrysis, Pyramimonas, Rhodomonas, Scenedesmus, Schizochytrium, Skeletonema, Spirulina, Synechococcus, Tetraselmis, Thalassiosira, Tisochrysis, and Tolypothrix. In some embodiments, the microalgae of the present disclosure are selected from the genera Chlorella. Scenedesmus, Nannochloropsis, Muriellopsis, Isochrysis, Tisochrysis, Desmodesmus, Haematococcus, Arthrospira, and Anabaena. In some embodiments, the compositions of the present disclosure comprise microalgae of a single genus or species. In some embodiments, the compositions of the present disclosure comprise microalgae of a consortia of microalgae genera or species.

Methods for culturing microalgae are known in the art. In some embodiments, the microalgae are grown according to conventional means for culturing microalgae. In some embodiments, initial microalgae strains and inoculum are generated and maintained in small volumes. Microalgae strains and cells intended for inclusion in the compositions can be selected based on the desired nutrient profile. In some embodiments, microalgae are grown through intensive and controlled culture of microalgae using photobioreactors. Photobioreactors allow the passage of light so that photosynthesis can occur while microalgae grow in optimized culture media. Any form of photobioreactor can be used to grow the microalgae of the present disclosure, include flat panel and tubular photobioreactors. Raceways may also be used for culturing microalgae. During microalgae growth, parameters such as pH, temperature, nutrients, dissolved oxygen and carbon dioxide injection can be maintained in order to ensure maximum production rates.

In some embodiments, microalgae are grown until biomass reaches 0.5-5.0 g/L. Microalgae are then harvested. In some embodiments, microalgae biomass is separated from the liquid culture, e.g., by centrifugation, settling, and/or filtration. Following separation of the biomass, the microalgae biomass is processed, in some embodiments, to ensure that microalgae are not living and/or to make available nutrients from within the microalgal cells. For example, in some embodiments, the biomass is dried. In some embodiments, the biomass is baked, dehydrated, desiccated, freeze-dried, and/or exposed to evaporative drying. In some embodiments, the microalgae is ground after drying to achieve a smaller particle size. In some embodiments, the dried microalgae is ground to a size of 1-10,000 microns. In some embodiments, the dried microalgae is ground to a size of 100-1,000 microns. A dried, ground composition of microalgae cells is referred to herein as “whole cell microalgae powder.” In some embodiments, a composition herein comprises 0.1-50 g/L of whole cell microalgae powder. In some embodiments, a composition herein comprises 0.8-20 g/L of whole cell microalgae powder.

In some embodiments, after separation of the biomass of the microalgae cells from the liquid solution, the microalgae is further processed to degrade cell walls and release nutrients, producing a digested microalgae solution or “DMS” of the present disclosure. Microalgae cells can be degraded by physical, mechanical, chemical, enzymatic, or biological means. In some embodiments, microalgae cells are physically disrupted, e.g., using high pressure and/or mechanical lysis. In some embodiments, microalgae cells are chemically disrupted, e.g., using acids. In some embodiments, microalgae cells are biologically disrupted, e.g., using enzymatic processes including proteolysis.

In some embodiments, the DMS has a nutrient profile as shown in FIG. 1. In some embodiments, humidity, e.g., water content, of DMS is about 75-95% w/w. In some embodiments, humidity is about 90% w/w. In some embodiments, dry matter is about 5-25% w/w. In some embodiments, dry matter is about 10% w/w. In some embodiments, the content of organic matter is about 5-20% w/w. In some embodiments, the content of organic matter is about 10% w/w. In some embodiments, the carbon content is about 1-15% w/w. In some embodiments, the carbon content is about 5% w/w. In some embodiments, the total nitrogen content of DMS is about 0.1-3.0% w/w. In some embodiments, the total nitrogen content of DMS is about 1-1.5% w/w. In some embodiments, the phosphorous content of DMS is about 0.05-0.5% w/w. In some embodiments, the phosphorous content of DMS is about 0.1% w/w. In some embodiments, the P₂O₅ content of DMS is about 0.05-0.5% w/w. In some embodiments, the P₂O₅ content of DMS is about 0.2% w/w. In some embodiments, the potassium content of DMS is about 0.1-1.0% w/w. In some embodiments, the potassium content of DMS is about 0.4% w/w. In some embodiments, the K₂O content of DMS is about 0.1-1.0% w/w. In some embodiments, the K₂O content of DMS is about 0.5% w/w. In some embodiments, the total nitrogen, phosphorous, and potassium (“NPK”) content including the weight of P₂O₅ and K₂O is about 0.5-5.0% w/w. In some embodiments, the total NPK content including the weight of P₂O₅ and K₂O is about 1.8% w/w. In some embodiments, the total amino acid content of DMS is about 1-15% w/w. In some embodiments, the total amino acid content of DMS is about 5% w/w. In some embodiments, the free amino acid content of DMS is 0.1-10% w/w. In some embodiments, the free amino acid content of DMS is about 2% w/w. In some embodiments, the density of DMS is about 1-1.1 g/mL. In some embodiments, the density of DMS is similar to that of water, i.e., around 1 g/mL. In some embodiments, the pH of DMS is acidic or is adjusted to be acidic. In some embodiments, the pH of DMS is or is adjusted to be about pH 3.5-pH 4.5. In some embodiments, the pH of DMS is adjusted to be around pH 6.0-6.5 or to match the pH of a carrier composition.

In some embodiments, the whole cell microalgae powder comprises the same amounts and/or ratios of components as DMS but with significantly less water content. In some embodiments, the whole-cell microalgae powder comprises less than 10% humidity by weight. In some embodiments, the whole-cell microalgae powder comprises less than 5% humidity by weight. In some embodiments, the whole-cell microalgae powder comprises 1-3% w/w humidity.

In some embodiments, the microalgae components of the present compositions comprise proteins, peptides, amino acids, plant hormones, phytohormones, carbohydrates, fatty acids, vitamins, minerals, polysaccharides, carotenoids, pigments, fibers, and other natural nutrients.

In some embodiments, the compositions disclosed herein differ from macroalgae and other biostimulant products in that the disclosed microalgae-derived compositions comprise a richer and more balanced biochemical composition. In some embodiments, the microalgae components of the present compositions provide all the essential free amino acids. In some embodiments, the microalgae components provide micronutrients, macronutrients, polyunsaturated fatty acids, antioxidants, carotenoids, and vitamins, as well as a high content and wide range of phytohormones. In some embodiments, the microalgae components help maintain the organic carbon in the soil and improve nutrient uptake. In some embodiments, the microalgae components provide a complete nutritional package to growing plants and help fight against abiotic stresses, improving the quality of the produce and the marketable yield.

In some embodiments, a composition of the disclosure, e.g., a granule composition, comprises 0.1%-10.0% w/w DMS. In some embodiments, a composition of the disclosure comprises 0.5%-5.0% w/w DMS.

In some embodiments, a composition of the disclosure, e.g., a liquid composition, comprises 10-100% w/w DMS. In some embodiments, a liquid composition comprising DMS is diluted to 0.3%-0.5% v/v in water prior to application.

In some embodiments, in terms of dry matter of microalgae, a composition comprises 0.01%-20% dry matter of microalgae. In some embodiments, in terms of dry matter of microalgae, a composition comprises 0.5%-5% dry matter of microalgae. In some embodiments, in terms of dry matter of microalgae, a composition comprises 0.05%-0.5% dry matter of microalgae. In some embodiments, in terms of dry matter of microalgae, a composition comprises 0.03%-0.05% dry matter of microalgae.

In some embodiments, a composition of the disclosure, e.g., a seed coating, comprises 5-95% w/w whole-cell microalgae powder. In some embodiments, a composition of the disclosure comprises 10-90% w/w whole-cell microalgae powder. In some embodiments, a composition of the disclosure comprises 20-80% w/w whole-cell microalgae powder.

In some embodiments, a composition of the disclosure, e.g., a liquid formulation, comprises 0.1-40 g/L whole-cell microalgae powder. In some embodiments, a composition of the disclosure, e.g., a liquid formulation, comprises 0.8-20 g/L whole-cell microalgae powder.

### Carriers

In some embodiments, the composition comprises a solid substrate or carrier. In some embodiments, carrier granules are prepared as a substrate or carrier for the combined solution. In some embodiments, granules are prepared prior to the mixture of the solution, or simultaneous with or after the solution preparation. In some embodiments, the carrier is a natural clay granule or mineral- or organic-based granule. In some embodiments, the carrier is limestone, silica, talc, kaolin, dolomite, calcium sulfate, calcium carbonate, magnesium sulfate, magnesium carbonate, magnesium oxide, diatomaceous earth, zeolite, bentonite, dolomite, leonardite, attapulgite, trehalose, chitosan, shellac, pozzolan, diatomite, or diatomaceous earth, or any combination thereof. In some embodiments, the carrier is a solid substrate formed as granules or extruded pellets of other materials such as synthetic fertilizer.

In some embodiments, the granules have a diameter of about 1-10 mm. In some embodiments, the granules have a diameter of about 2-4 mm.

Natural clay based granules are inert, biodegradable, resistant to attrition due to mixing, and have a neutral pH. Accordingly, in some embodiments, the acidity of a coating solution is matched to that of the carrier prior to coating. Clay granules are available in several size grades from 12/25 mesh to 10/20 & 16/35 mesh (ASTM). A range of carrier sizes are suitable for use in some embodiments of the disclosure.

In some embodiments, the granules are formed from zeolite. Zeolite is a soil conditioner that can control and raise the pH of the soil and improve soil moisture. Synthetic and natural zeolites are hydrated aluminosilicates with symmetrically stacked alumina and silica tetrahedra which result in an open and stable three-dimensional honeycomb structure with a negative charge. The negative charge within the pores is neutralized by positively charged ions (cations) such as sodium. Their aluminosilicate frameworks allows them to be used as cationic exchangers because of their high cation exchange capacity (CEC) due to the presence of trivalent Al atoms in the zeolite framework which induce negative charges that are compensated by the presence of cations. In some embodiments, the zeolite is a natural zeolite. In some embodiments, the zeolite is a synthetic zeolite. In some embodiments, the zeolite is Clinoptilolite.

In some embodiments, the granules are formed from dolomite. Dolomite can be used for soil neutralization to correct acidity. Adding zeolite or dolomite to manure improves the nitrification process. These materials are commonly used as slow release substances for pesticides, herbicides and fungicides. In some embodiments, zeolite or dolomite particles, or combinations of the two, may be used for the carrier granules.

In some embodiments, attapulgite is used as the carrier granule. Attapulgite is a magnesium aluminum phyllosilicate which occurs in a type of clay soil, and it is used as a processing aid and functions as a natural bleaching clay for the purification of vegetable and animal oils. It is available in both colloidal and non-colloidal forms. In some embodiments, attapulgite particles or granules are used as carrier granules in the present compositions.

Leonardite is an oxidation product of lignite coal, mined from near surface pits. Leonardite is a high quality humic material soil conditioner which acts as a natural chelator. It is typically soft, dark colored, and vitreous, containing high concentrations of the active humic acid and fulvic acid. In some embodiments, leonardite is used, alone or in combination with other materials, as a carrier granule.

Bentonite pellets are used in agriculture for soil improvement, livestock feed additives, pesticide carriers, and other purposes. Bentonite mixed with chemical fertilizer can fix ammonia and can act as a buffer for fertilizers. The inherent characteristics of water retention and absorbency makes it an ideal addition to improve the fertility of soil. The prevalence of sandy soil in many regions that suffer from low water and nutrient holding characteristics, can be significantly enhanced by the addition and blending of calcined bentonite. In some embodiments, bentonite, or calcined bentonite, is used as a carrier granule.

In some embodiments, the carrier granules comprise a mix of different materials such as clay, leonardite, attapulgite, zeolite, and/or bentonite.

In some embodiments, the composition comprises more than 50% w/w solid carrier. In some embodiments, the composition comprises more than 70, 80, 90, or 95% w/w solid carrier. In some embodiments, the composition comprises about 80-95% w/w solid carrier.

In some embodiments, the composition comprises a liquid carrier. Non-limiting examples of liquids useful as carriers for compositions disclosed herein include water, an aqueous solution, or a non-aqueous solution. In some embodiments, a carrier is water. In some embodiments, a carrier is an aqueous solution. In some embodiments, a carrier is a non-aqueous solution. For example, in embodiment involving a soil drench, foliar spray, or other liquid composition, suitable liquid carriers include water, buffered water, and oils.

In some embodiments, the composition comprises more than about 90% w/w liquid carrier. In some embodiments, the composition comprises about 95-99.9% w/w liquid carrier. In some embodiments, the composition comprise about 99.5-99.7% w/w liquid carrier.

### Additional Ingredients

In some embodiments, the composition comprises ingredients in addition to CFS, microalgae and mycorrhizae components. In some embodiments, the composition comprises an excipient, surfactant, diluent, binder, disintegrant, inert filler, pH stabilizer, spreader, fixative, defoamer, carrier, antimicrobial agent, fertilizer, nutrient additive, pesticide, herbicide, fungicide, insecticide, nematicide, molluscicide, antifreeze agent, antioxidant, preservative, or anti-aggregation agent. In some embodiments, the composition comprises a biostimulant. In some embodiments, the composition comprises a plant growth regulator. One of ordinary skill in the art will appreciate that additional agrochemically acceptable excipients are available for inclusion in the present compositions without departing from the scope of the disclosure. Agriculturally acceptable excipients are commercially manufactured and available through a variety of companies.

In some embodiments, the composition comprises a binder. In some embodiments, the composition comprises a hydrocolloid. In some embodiments, the composition comprises a vinasse, lignosulfonate, cellulose, anhydrite, sugar, starch, or clay.

In some embodiments, the composition is mixed with one of the aforementioned additional ingredients. In some embodiments, the composition is administered at the same time as one of the aforementioned additional ingredients. In some embodiments, the composition is administered shortly before or shortly after one of the aforementioned additional ingredients.

### Methods of Using Compositions Comprising Microalgae

The present disclosure provides methods of using the compositions described herein on a host plant.

### Host Plants

The methods of the present disclosure may be used on any host plant. In some embodiments, the host plant is an agricultural crop. Agricultural crops include agronomic crops, horticultural crops, and ornamental plants. In some embodiments, a method of the present disclosure is employed on an agronomical crop selected from the list consisting of wheat, rice, corn, soy bean, alfalfa, forage crops, beans, sugar beets, canola, and cotton. In some embodiments, a method of the disclosure is employed on a horticultural crop selected from the list consisting of vegetables, fruits, flowers, ornamentals, and lawn grasses. In some embodiments, a method of the disclosure is employed on an ornamental plant selected from the list consisting of flowers, shrubs, grasses, and trees.

Host plants include both monocots and dicots. In some embodiments, the methods of the disclosure are employed on monocots, such as agapanthus, asparagus, bamboo, bananas, corn, daffodils, garlic, ginger, grass, lilies, onions, orchids, rice, sugarcane, tulips, and wheat. In some embodiments, the methods of the disclosure are employed on dicots, such as apples, beans, broccoli, carrots, cauliflower, cosmos, daisies, peaches, peppers, potatoes, roses, sweet pea, and tomatoes. In some embodiments, the agricultural crop is a food crops, feed crop, cereal crop, oil seed crop, pulse, fiber crop, sugar crop, forage crop, medicinal crop, root crop, tuber crop, vegetable crop, fruit crop, or garden crop.

Compositions of the present invention may be applied to any plant or plant propagation material that may benefit from improved growth including agricultural crops, annual grasses, trees, shrubs, ornamental flowers and the like.

In some embodiments, the agricultural crop is selected from cereals, plantation crops, groundnut crops, grams, pulses, vegetables, fruits, proteaginous crops, citrus crops, berry crops, melon crops, vine crops. In some embodiments, the agricultural crop is selected from the list consisting of apple, barley, sunflower, plum, rice, paddy rice, agave, strawberry, watermelon, coffee, tomato, lentil, pea, chickpea, potato, cotton, sugarcane, wheat, banana, soy bean, corn, sorghum, onion, carrot, bean, zucchini, lettuce, chicory, fennel, sweet pepper, pear, peach, cherry, kiwifruit, soft wheat, durum wheat, grapevine, table grape, olive, almond, hazelnut, cotton, canola, and maize.

### Application Methods and Application Rates

In some embodiments, the methods comprise applying a dry granule formulation as described herein. The dry granule formulation can be applied to the crops by any suitable means. In some embodiments, the granules are broadcast onto the soil, e.g., by hand or by machine. In some embodiments, the granules are pre-mixed with sand, soil, and/or fertilizer before broadcast. In some embodiments, the compositions are spread, brushed, or sprayed onto the crops or the environs thereof by hand, by apparatus, or by machine. In some embodiments, the dry granule formulation is applied at the rate of 1-100 kg per hectare. In some embodiments, the dry granule formulation is applied at the rate of 5-50 kg per hectare. In some embodiments, the dry granule formulation is applied at the rate of about 10 kg per hectare.

In some embodiments, the present methods comprise applying a seed coating as described herein. In some embodiments, the seed coating is applied to the seeds before planting. e.g., using a mixer. In some embodiments, the seed coating is applied in furrow, e.g., via suitable broadcast or in-furrow application means. In some embodiments, the seed coating is applied using flow equipment after suspension in a liquid carrier. In some embodiments, the seed coating is applied at the rate of about 10 g to 1 kg of dry powder seed coating per quantity of seeds to be planted in one hectare. In some embodiments, the seed coating is applied at the rate of about 50-200 g of dry powder seed coating per quantity of seeds to be planted in one hectare. In some embodiments, the seed coating is applied at the rate of about 100 g of dry powder seed coating per quantity of seeds to be planted in one hectare.

In some embodiments, the present methods comprise applying a liquid formulation as described herein. In some embodiments, the liquid formulation is applied at a rate of 100 mL to 100 L per hectare. In some embodiments, the liquid formulation is applied at a rate of 0.5 L to 10 L per hectare. In some embodiments, the liquid formulation is applied at a rate of about 4-7 L per hectare. In some embodiments, the liquid formulations herein are diluted in water or a suitable liquid carrier prior to application. For example. In some embodiments, the liquid formulations are diluted to 0.1-1.0% v/v before application to the host plant, plant parts, or plant environs. In some embodiments, the liquid formulations are diluted to 0.3-0.5% v/v before application.

The compositions of the present disclosure may be applied to any part of a host plant or the environs thereof. In some embodiments, in the case of granules, the compositions are applied to the roots and/or the soil around the host plant. In some embodiments, in the case of seed coatings, the compositions are applied to the seeds of the host plant before, during or shortly after planting. In the case of liquid compositions, the compositions may be applied to the seeds, seedlings, plants, or plant parts. Plant parts include seeds, seedlings, plant tissues, leaves, branches, stems, bulbs, tubers, roots, root hairs, rhizomes, cuttings, flowers, and fruits. Compositions of the present invention may further be applied to any area where a plant will grow including soil, a plant root zone and a furrow.

The compositions of the present disclosure can be applied at any time during the host plant life cycle. In some embodiments, the compositions of the present disclosure are applied shortly after planting, tillering, or sowing. In some embodiments, the compositions of the present disclosure are applied as a seed coating or soil treatment around the time of planting. In some embodiments, the compositions are applied 0-30 days after planting, sowing, or tillering. In some embodiments, the compositions are applied pre-blooming. In some embodiments, the compositions are applied post-blooming. In some embodiments, the compositions are applied at rooting, sprouting, flowering, fruit setting, ripening, or fattening. In some embodiments, the compositions are applied before or during a peak period of metabolic activity. In some embodiments, the compositions are applied during a period of host plant stress.

In some embodiments, the compositions are applied more than once. In some embodiments, the composition is administered 3 to 5 times per growing cycle, depending on the type of crop, the intensity, and the planting. In some embodiments, the compositions are applied periodically throughout the growing cycle. The compositions may be applied once a day, once a week, once every two weeks, or once a month. In some embodiments, the timing of composition application is based on field studies assessing the efficacy of application at different time points. In some embodiments, the compositions are applied 1-10 times throughout the growing cycle of the host plant. In some embodiments, the compositions are applied 1-5 times throughout the growing cycle of the host plant.

In some embodiments, application to plants, plant parts, plant tissues, or plant environs comprises soil application pre-blooming and application to aerial biomass post-blooming. In some embodiments, compositions intended for soil are applied pre-blooming, such as granules or liquid soil treatments, and compositions intended for aerial dispersion are applied post-blooming, such as foliar sprays.

### Improving Host Plant Nutrient Utilization

The present disclosure provides methods for improving host plant nutrient utilization. The methods comprise applying a microalgae-based composition of the present disclosure to the host plant.

In some embodiments, the method upregulates gene expression in the host plant. In some embodiments, the method upregulates host plant expression of a gene involved in nitrogen metabolism. In some embodiments, the method upregulates expression of a gene selected from nitrate reductase, glutamine synthetase, glutamate synthase, glutamic acid synthase, and glutamate dehydrogenase. In some embodiments, the method upregulates expression of nitrate reductase. In some embodiments, the method upregulates expression of glutamate dehydrogenase. In some embodiments, the method upregulates expression of glutamate synthase.

In some embodiments, the method upregulates host plant expression of a gene involved in amino acid metabolism. In some embodiments, the method upregulates expression of a gene involved in the biosynthesis of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine. In some embodiments, the method upregulates expression of a gene involved in alanine, aspartate, or glutamate metabolism. In some embodiments, the method upregulates expression of alanine:glyoxylate aminotransferase (AGT), an N-terminal nucleophile aminohydrolase, L-aspartate oxidase, alanine aminotransferase 2 (ALAAT2), a pyridoxal phosphate (PLP)-dependent transferase, glutamine-dependent asparagine synthase 1 (ASNI), or glutamate dehydrogenase 2 (GDH2). In some embodiments, the method upregulates the expression of one or more of AGT, ALAAT2, ASNI, or GDH2.

### Decreased Reliance on Exogenous Nitrogen Supplementation

In some embodiments, the improved nutrient utilization of the host plant leads to an increase in a growing parameter or production parameter of a host plant. In some embodiments, the improved nutrient utilization of the host plant leads to an increase in a growing parameter or production parameter of a host plant compared to a control plant without the application of the microalgae composition. In some embodiments, improved nutrient utilization leads to a decreased dependence on exogenous nitrogen application. In some embodiments, the methods allow for 1-50% less exogenous nitrogen application to the host plant during the growing cycle, without negatively impacting a parameter, e.g. a production parameter, of the host plant. In some embodiments, the methods allow for 5%, 10%, 15%, 20%, 25%, 30%, 35%, 40%, 45%, or 50% less exogenous nitrogen application to the host plant during the growing cycle, without negatively impacting a parameter, e.g. a production parameter, of the host plant. In some embodiments, the host plant attains the same or better parameter value than a control untreated plant with less exogenous nitrogen application to the host plant. In some embodiments, the host plant outperforms a control plant receiving the same amount of exogenous nitrogen. In some embodiments, the host plant outperforms a control plant, wherein the control plant is receiving a greater quantity of exogenous nitrogen.

In some embodiments, the method increases a growing parameter of the host plant with less need for external nutrient supplementation, e.g., exogenous nitrogen supplementation. A growing parameter is related to the growth of the host plant. Growing parameters include plant size, biomass (dry or wet), aerial biomass, height, number of branches, number of leaves, number of flowers, root biomass, number of roots, number of secondary roots, root volume, root length, and degree of inoculation by diazotrophic bacteria.

In some embodiments, the method increases a production parameter of the host plant with less need for external nutrient supplementation, e.g., exogenous nitrogen supplementation. A production parameter is related to the plant part that is harvested from the plant for commercial purposes. Production parameters include, but are not limited to, yield, yield per plant, yield per area harvested biomass, harvested weight, harvested volume, number of harvested plant parts, and size of harvested plant parts. In terms of the harvestable plant parts, production parameters include yield, weight, size, and number of harvestable plant parts. Harvestable plant parts include, for example, fruits, vegetables, roots, grains, tubers, leaves, flowers, seeds, and nuts. In some embodiments, e.g., for some grasses, lettuces, feed crops, and forage crops, a harvestable plant part is the entire aerial biomass of the plant. In some embodiments, the harvestable plant part is related to the intended use of the crop. For example, for oil crops, the harvestable plant parts are the components of the plant containing the oil to be harvested.

In some embodiments, the method improves a growing parameter or production parameter compared to a control condition. In some embodiments, the method improves a parameter in terms of timing. i.e., the parameter is improved at a given time point compared to the control. For example, in some embodiments, the method may improve a growing parameter relative to a control early on, such as early flowering, faster maturation, increased height compared to control at the same time point.

Improving Soil Fitness and/or Rhizospheric Activity

In one aspect, the present disclosure provides methods of improving soil fitness. Soil fitness includes soil-specific parameters such as: macronutrient content, micronutrient content, phosphorous cycling, nitrogen cycling, carbon cycling, enzymatic activity, metabolic activity, microbial population size, and microbial diversity. Examples of soil fitness parameters increased by the present methods include organic carbon content, water soluble carbon content, water soluble polyphenols, humic substance content, total nitrogen content, water soluble nitrogen content, nitrate content, assimilable phosphorous content, alkaline phosphatase activity, beta-glucosidase activity, urease activity, basal respiration, concentration of microorganisms, diversity of microorganisms, concentration of bacteria, concentration of gram positive bacteria, concentration of gram negative bacteria, concentration of fungi, preferential development of gram positive bacteria, and the diversity of carbon source utilization within the microbiome.

In some embodiments, the methods increase the concentration of a macronutrient in the soil. In some embodiments, the methods increase the concentration of a micronutrient within the soil. In some embodiments, the methods increase the concentration of a host-plant available micronutrient or macronutrient within the soil. In some embodiments, the methods increase the concentration of a water soluble micronutrient or macronutrient within the soil. In some embodiments, the methods increase the carbon, nitrogen, or phosphorous content of the soil. In some embodiments, the methods increase carbon, nitrogen, or phosphorous cycling within the soil. In some embodiments, the methods increase the metabolic activity taking place within the soil. In some embodiments, the methods increase microbial concentration and/or microbial diversity within the soil.

In some embodiments, surprisingly, the methods increase the soil fitness, including increasing macronutrient and/or micronutrient concentration without affecting the macronutrient and/or micronutrient composition of the leaves of a plant growing within the soil. In some embodiments, the methods provide beneficial effects on soil quality that improve a plant parameter, such as a growing parameter, a production parameter, or a biostimulant parameter, without affecting the plant leaf composition.

In some embodiments, the soil fitness parameter is related to rhizospheric growth and/or rhizospheric activity. In some embodiments, the methods of the present disclosure increase rhizospheric growth and/or activity within the soil. Rhizospheric parameters that can be increased by the methods of the disclosure include: phosphorous cycling, nitrogen cycling, carbon cycling, enzymatic activity, metabolic activity, microbial population size, and microbial diversity. Specific rhizospheric parameters that can be increased by the present methods include: alkaline phosphatase activity, beta-glucosidase activity, urease activity, basal respiration, concentration of microorganisms, diversity of microorganisms, concentration of bacteria, concentration of gram positive bacteria, concentration of gram negative bacteria, concentration of fungi, preferential development of gram positive bacteria, and the diversity of carbon source utilization within the microbiome.

In some embodiments, a soil fitness parameter is increased compared to a control soil without microalgae composition application. In some embodiments, a soil fitness parameter is increased by 1-200% compared to a control soil. In some embodiments, a soil fitness parameter is increased by 10-100% compared to a control soil. In some embodiments, a soil fitness parameter is increased by 2-fold to 10-fold compared to a control soil. In some embodiments, a soil fitness parameter is increased at a particular time point compared to the control. For example, in some embodiments, application of the microalgae compositions of the disclosure improve a soil fitness parameter compared to control at maturation, post harvest, blooming, sprouting, or fattening. In some embodiments, the soil microbial diversity is increased 2 to 10-fold compared to control. In some embodiments, the soil microbial diversity is increased 5-fold compared to control.

Improvements in Host Plant Response to and Recovery from Abiotic Stress

The present disclosure provides methods of improving a host plant's resistance to abiotic stress by applying a composition of the disclosure to the host plant. The present disclosure also provides methods of improving a host plant's recovery to abiotic stress by applying a composition of the disclosure.

“Improving resistance to abiotic stress” as used herein refers to decreasing the damage sustained by a host plant in response to exposure to abiotic stress. Damage may be evaluated in the short term (e.g., wilting) or in the long term (e.g., loss of yield). Damage from abiotic stress includes a decrease in a growing parameter, production parameter, or biostimulant parameter of a host plant following exposure to the abiotic stress. Short term damage from abiotic stress includes, but is not limited to, discoloring, wilting, yellowing, leaf curling, leaf browning, spots on leaves, dropped leaves, dropped flowers, deformity, and reduced vigor. Long term damage from abiotic stress includes, but is not limited to, stunted growth, plant death, decreased plant height, decreased plant volume, reduced productivity, reduced yield, reduced number of fruit, and reduced quality of fruit.

Abiotic stress includes water stress, temperature stress, sun stress, salinity stress, wind stress, herbicidal stress, and heavy metal stress. Examples of abiotic stress include drought, heat, cold, excess salinity, herbicide exposure, strong winds, heavy metals, flooding, and excessive sunlight.

In some embodiments, the present methods improve resistance to abiotic stress. In some embodiments, the present methods improve resistance to temperature stress. In some embodiments, the present methods improve resistance to water stress. In some embodiments, the present methods improve resistance to salinity stress. In some embodiments, the present methods improve resistance to sun stress. In some embodiments, the present methods improve resistance to wind stress. In some embodiments, the present methods improve resistance to heavy metal stress. In some embodiments, the present methods improve resistance to herbicidal stress.

In some embodiments, the present methods improve recovery from abiotic stress. As used herein “improved recovery from abiotic stress” refers to an improved recovery from the damage incurred as a result of exposure to abiotic stress, after the abiotic stress exposure has ceased. In some embodiments, improved recovery is evaluated in terms of the degree of recovery. In some embodiments, improved recovery is evaluated in terms of decreased time to recover a normal growing parameter, production parameter, or biostimulant parameter. In some embodiments, improved recovery is evaluated over time in comparison to an untreated control plant. In some embodiments, the treated plant exhibits improved recovery compared to the control plant in terms of a growing parameter, production parameter, or biostimulant parameter at a given time point after the abiotic stress exposure has ceased. Recovery from abiotic stress includes, but is not limited to, recovery from wilting, discoloration, spotting, curling, drooping, reduced growth, reduced leafing, and reduced flowering.

In some embodiments, the present methods improve recovery from temperature stress. In some embodiments, the present methods improve recovery from water stress. In some embodiments, the present methods improve recovery from salinity stress. In some embodiments, the present methods improve recovery from sun stress. In some embodiments, the present methods improve recovery from wind stress. In some embodiments, the present methods improve recovery from heavy metal stress. In some embodiments, the present methods improve recovery from herbicidal stress.

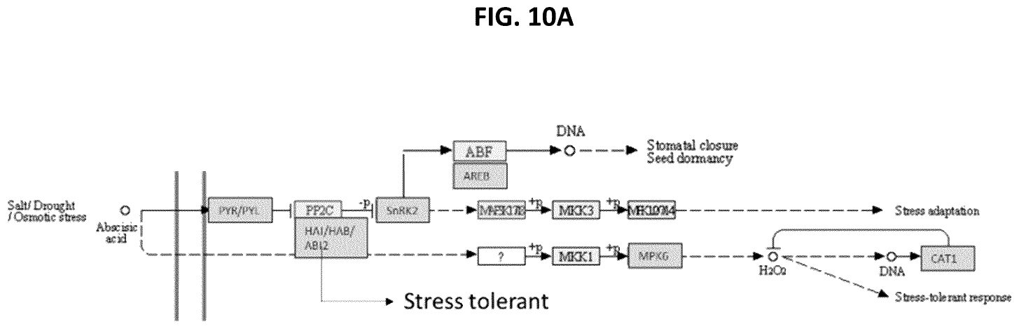

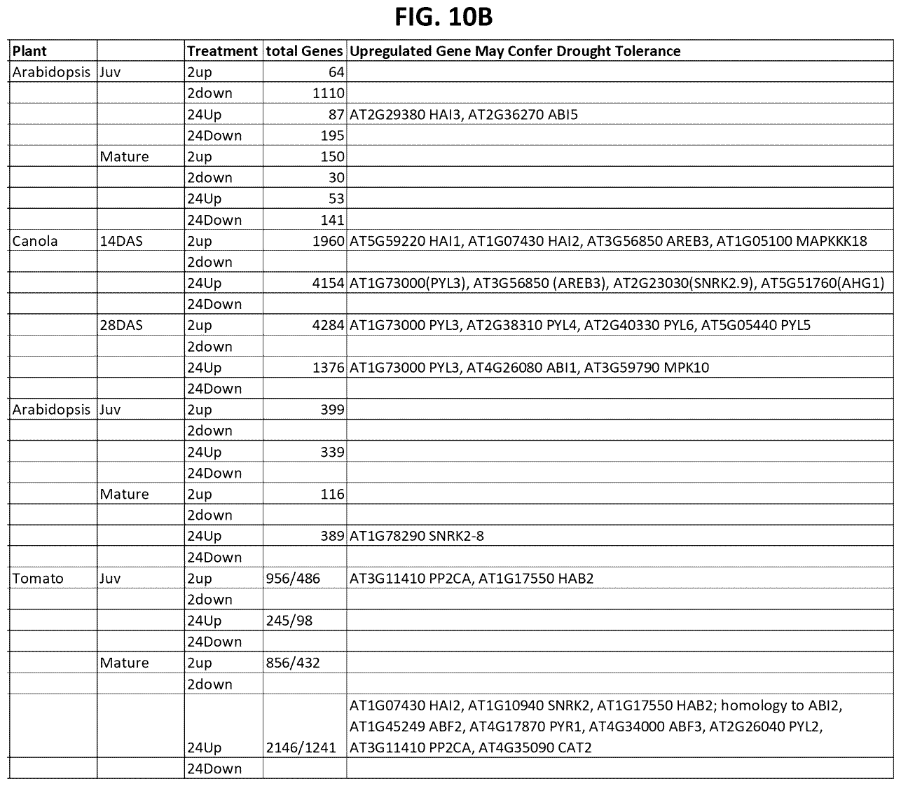

In some embodiments, the method upregulates expression of genes involved in response to abiotic stress. In some embodiments, the method upregulates expression of genes involved in response to abiotic stimulus, response to water deprivation, or response to stress. In some embodiments, the method improves host plant nutrient utilization, thereby allowing the host plant to better tolerate abiotic stress. In some embodiments, the method upregulates expression of a gene encoding an ABA receptor, a protein phosphatase 2C, an SNF1-related protein kinase 2, an ABRE-binding protein, an ABRE-binding factor, or a mitogen-activated protein kinase. In some embodiments, the method upregulates expression of a PYL, PP2C. SnRK2, AREB, ABF, or MAPK gene. In some embodiments, the method upregulates expression of any one or more of the following genes, as identified in FIG. 10B based on Arabidopsis gene model: ABF2, ABF3, ABI1, ABI2, ABI5, AHGI, AREB3, CAT2, HAB2, HAI1, HAI2, HAI3, MAPKKK18, MPK10, PP2CA, PYL2, PYL3, PYL4, PYL5, PYL6, PYRI, SNRK2, SNRK2.9, SNRK2-8, and homologs thereof.

### EXAMPLES

### Example 1: Formulation of Illustrative Components of Compositions of the Disclosure

Whole-cell microalgae powder. A microalgae consortium comprising genera from the list of Chlorella, Scenedesmus, Nannochloropsis, Muriellopsis, Isochrysis, Tisochrysis, Desmodesmus, Haematococcus, Arthrospira, and Anabaena was cultured in photobioreactors supplemented with nutrients and CO2. The microalgae were harvested once the biomass reached 0.5-5.0 g/L. Culture solids comprising whole microalgae cells were then separated from solution, dried, and ground to an average particle size of about 100-1000 microns in order to produce a mostly whole cell powder form of microalgae, i.e., “whole-cell microalgae powder.”

Digested microalgae solution. The whole cell microalgae powder was then processed to degrade cell walls and proteins, thereby increasing the concentration of accessible organic carbon, amino acids and peptides and producing a digested microalgae solution (“DMS”) of the disclosure. A nutrient analysis of an illustrative DMS is shown in FIG. 1.

Liquid microalgae applications. For liquid microalgae applications, e.g., in the form of foliar sprays, the DMS was typically diluted to 0.3-0.5% v/v with demineralized water and optionally a buffer.

### Example 2: Improved Host Plant Nutrient Utilization with Application of Microalgae Composition

### Materials and Methods

Tomato plants were grown with a standard rate of exogenous nitrogen supplementation or 75% rate of exogenous nitrogen supplementation and with or without DMS, formulated as in Example 1. DMS was applied in 2 soil applications at 4 L/ha pre-blooming and 3 foliar applications at 4 L/ha post-blooming. The DMS was diluted to 0.3-0.5% v/v before application. Each plot area comprised 5×40′ and there were four replicates per treatment condition. Each treatment condition was otherwise grown according to grower standard conditions.

### Results

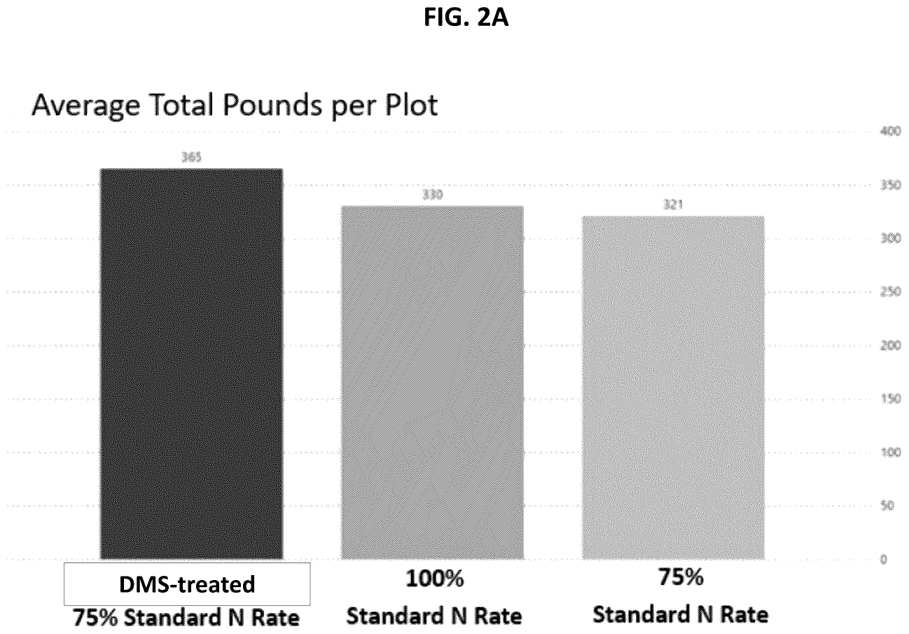

Application of an illustrative microalgae composition of the disclosure resulted in increased overall yield, improved fruit size, and decreased nitrogen fertilizer stress compared to the grower standard. FIG. 2A shows the tomato yield results in average total pounds per plot of application in each treatment condition. DMS with 75% exogenous nitrogen outperformed the control with 100% and 75% nitrogen. There was a 10.6% increase in total yield compared to the 100% control and a 13.7% increase in total yield compared to the 75% control. FIG. 2B shows the results in terms of number of large, medium, and small tomatoes between DMS treated and untreated conditions with 75% nitrogen. The yield of large tomatoes was 58% higher in the DMS treated condition.

### Example 3: Genes Upregulated in Amino Acid and Nitrogen Metabolism after Application of Illustrative Microalgae Composition

### Materials and Methods

Arabidopsis and tomato RNAseq experiment. 0.3% DMS was applied to juvenile and mature Arabidopsis thaliana and juvenile and mature Solanum lycopersicum tomato plants, which were compared to control untreated plants. RNAseq was performed on plant leaves 2 hours and 24 hours after application. For Arabidopsis, the data quality was excellent with 99.54% of reads retained after read trimming and more than 95% of reads mapped for most samples using the Gydle nuclear algorithm. Over 25,000 transcripts were mapped per sample, about 52% of total. For tomato, The ITAG3.2 genomes and annotations were used in this analysis as reference genomes. Short reading mapping, transcript assembly, and quantification were performed using the HISAT2 and Stringtie software tools. Gene expression was estimated as FPKM (Fragments Per Kilobase of exons and Million of mapped reads). The number of transcripts per gene was assessed in each of the samples and averaged among the replicates in the same treatment condition.

Up regulated genes were defined as those for which the average number of transcripts in the treated plant divided by the average value for control was greater than 2, and the average number of transcripts in the treated condition was over 10. Down regulated genes were defined as those for which the average value for treatment divided by the average value for control was less than 0.5, and the average value for control was over 10.

GO term enrichment was assessed using the Panther Gene Ontology database by identifying the number of genes for that GO term that were identified in the treated sample, and comparing that to the expected number given the total number of genes identified in the sample and the total number of genes belonging to that GO term in the genome.

### Results

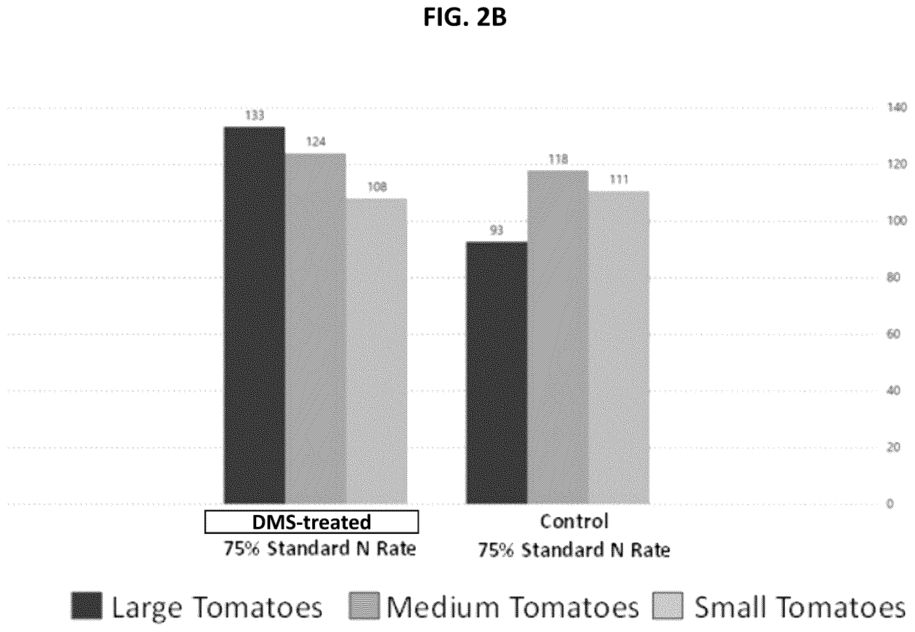

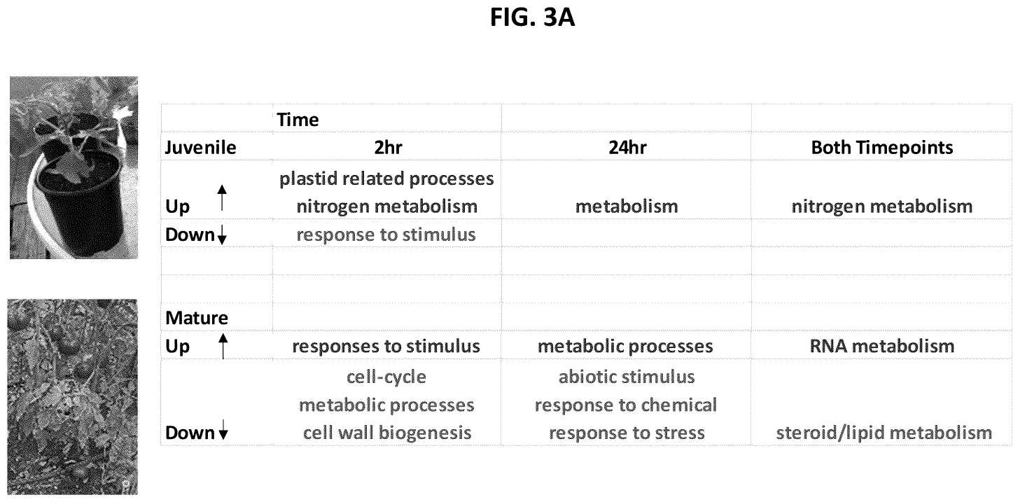

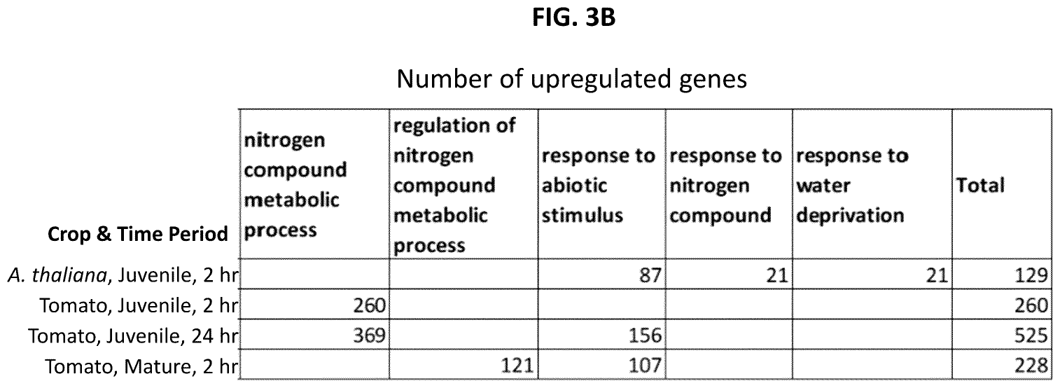

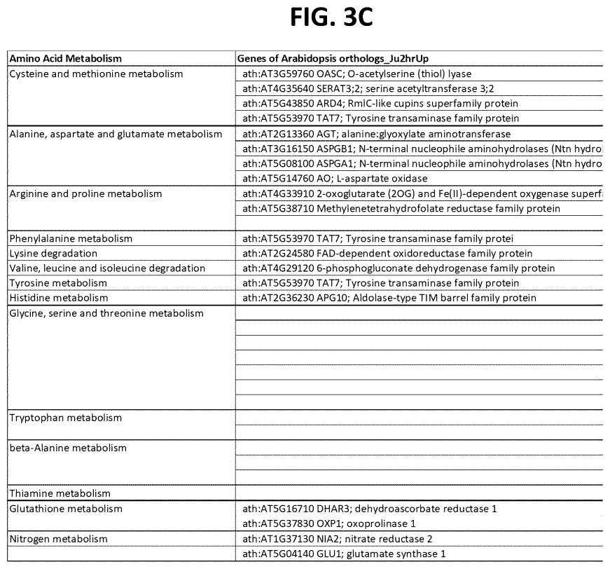

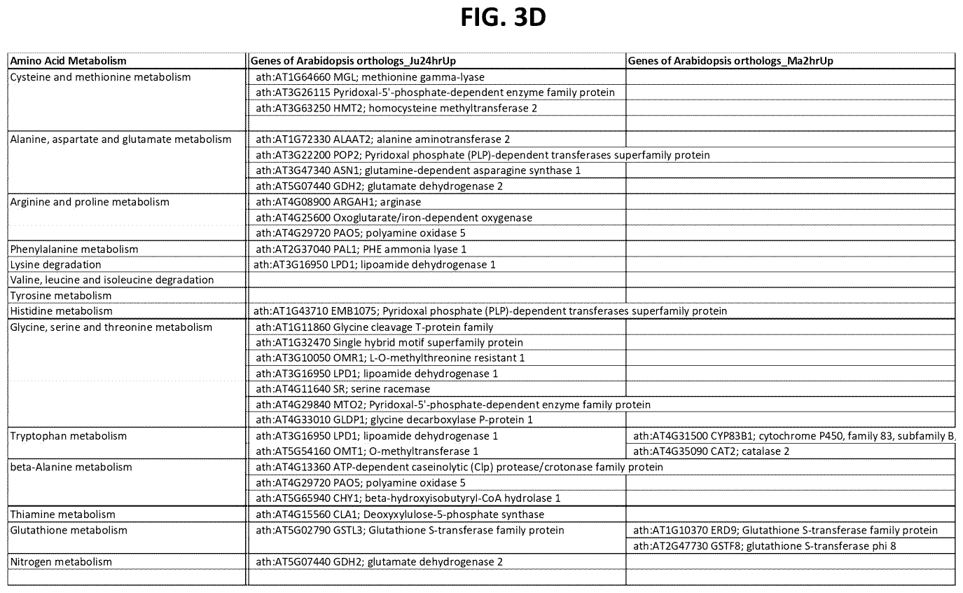

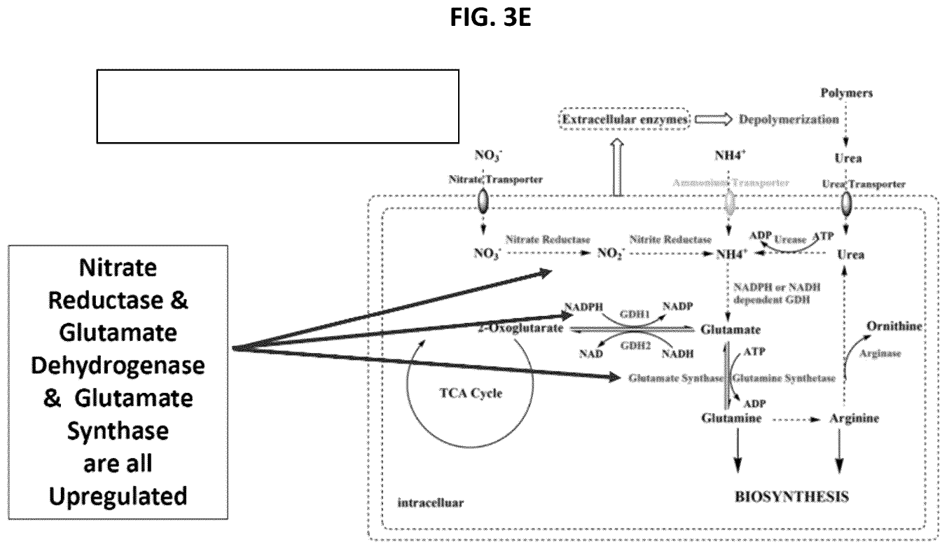

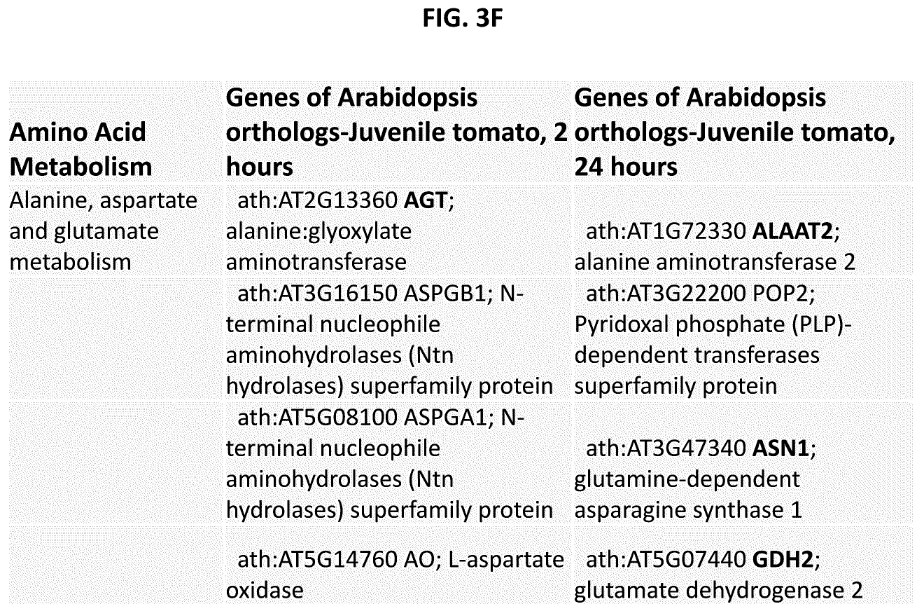

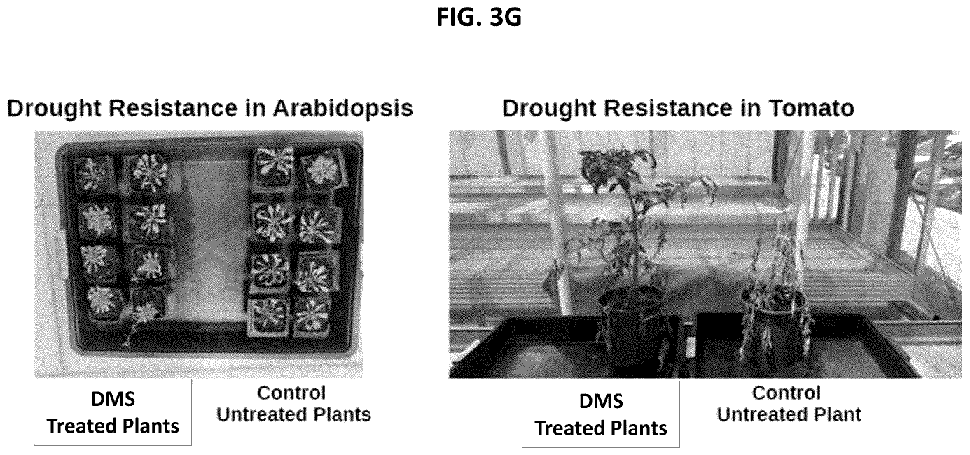

Treatment with an illustrative microalgae composition of the disclosure elicited large and different transcriptomic changes in both juvenile and mature Arabidopsis and tomato plants at both 2 and 24 hour time points. In both juvenile and mature tomato leaves, genes involved in metabolism were upregulated (FIG. 3A). FIG. 3B shows examples of gene categories and number of identified genes in different treatment groups-genes involved in nitrogen metabolism and responses to abiotic stress were upregulated in these conditions. Examples of genes related to amino acid and nitrogen metabolism are shown for juvenile tomato plants at 2 hours (FIG. 3C), juvenile tomato plants at 24 hours (FIG. 3D), mature tomato plants at 2 hours (FIG. 3D). Among the upregulated genes were nitrate reductase, glutamate dehydrogenase, glutamate synthase, alanine:glyoxylate aminotransferase, and alanine aminotransferase 2, which are involved in nitrogen and amino acid metabolism (FIG. 3E-3F).

The improved response to abiotic stress and improved nutrient utilization was validated in drought resistance assays in Arabidopsis and tomato plants, in which DMS-treated plants were observed to demonstrate higher drought tolerance compared to control. See FIG. 3G. Also see Example 5.

### Example 4: Microalgae Composition Application Improves Soil Fitness, Microbial Activity and Diversity

### Materials and Methods

DMS was applied to the soil of young (6 yo) and adult (30 yo) almond trees at five time points over the course of a year: maturation (T1), post harvest (T2), blooming (T3), sprouting (T4), and fattening (T5). Measurements of soil parameters were made before any of the applications (T0) and at the different time points after DMS application. Soil was evaluated for carbon content, nitrogen content, phosphorous content, enzymatic activity, microbial activity, and microbial diversity.

### Results

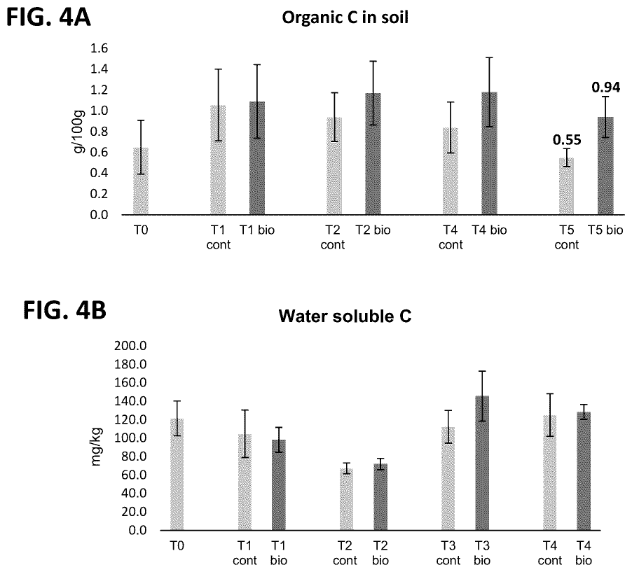

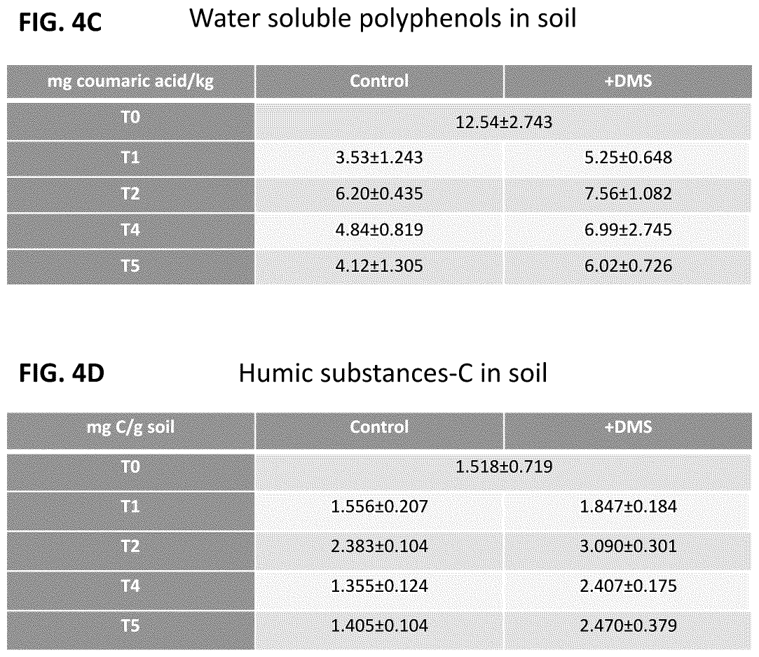

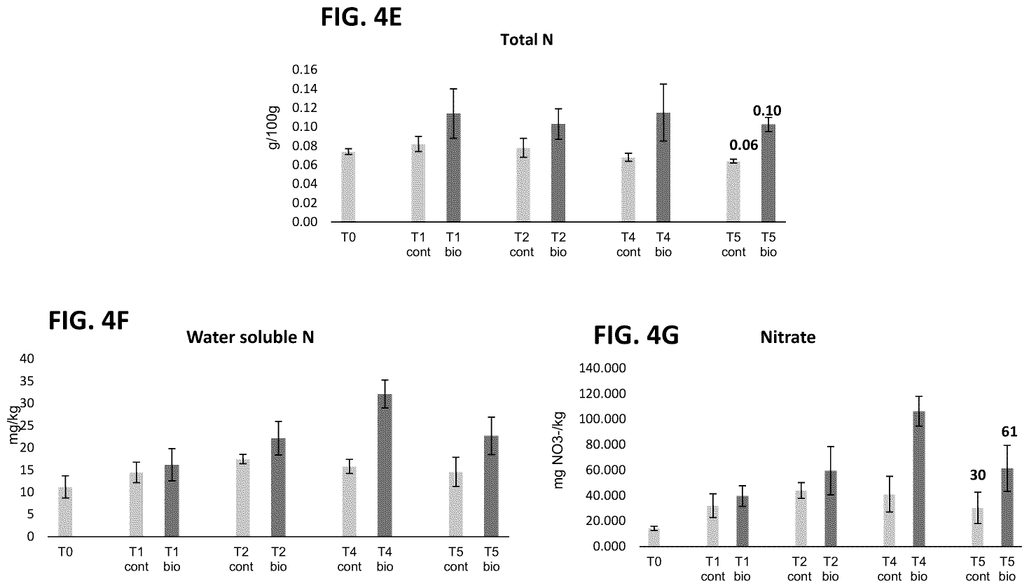

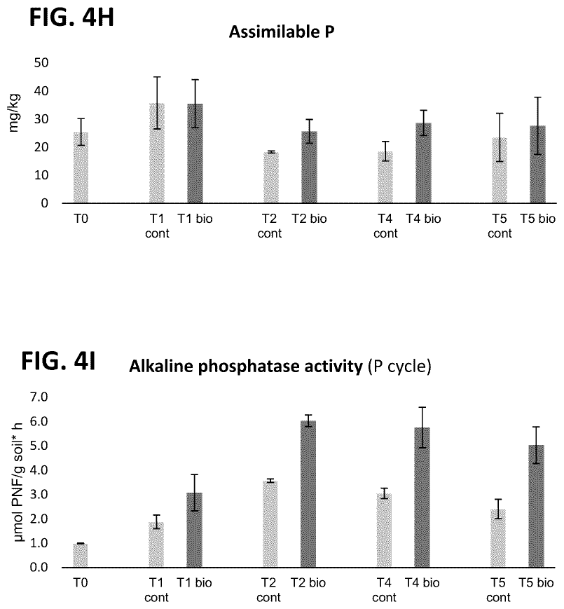

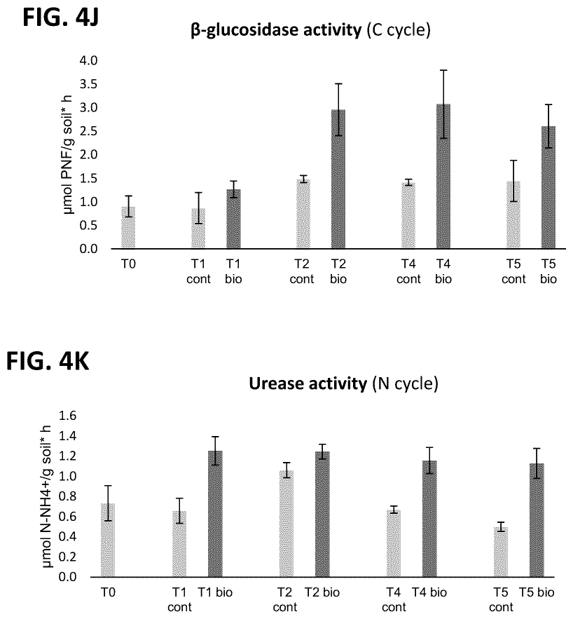

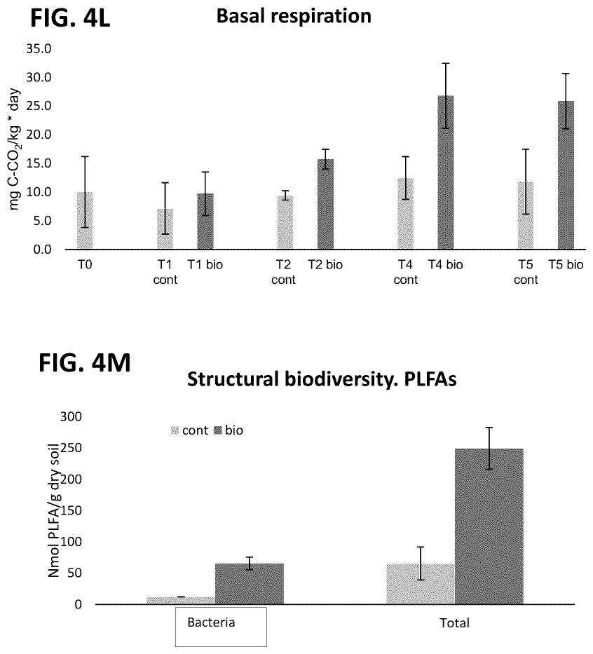

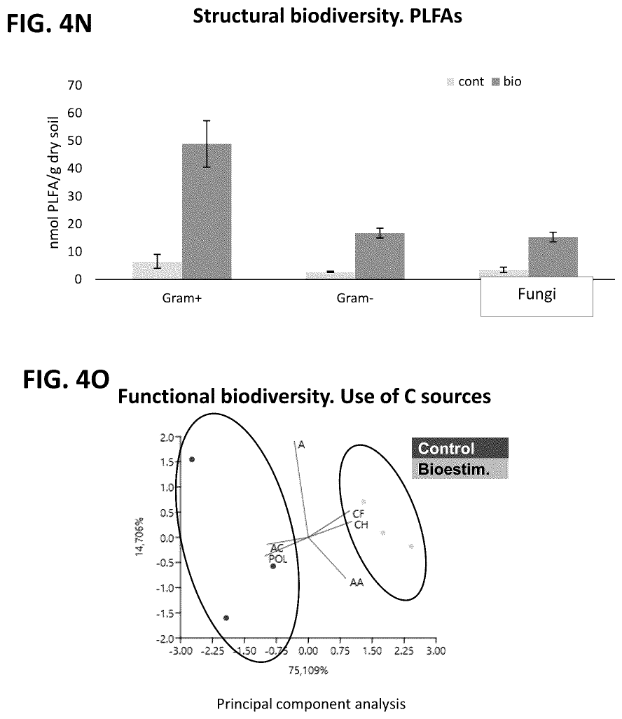

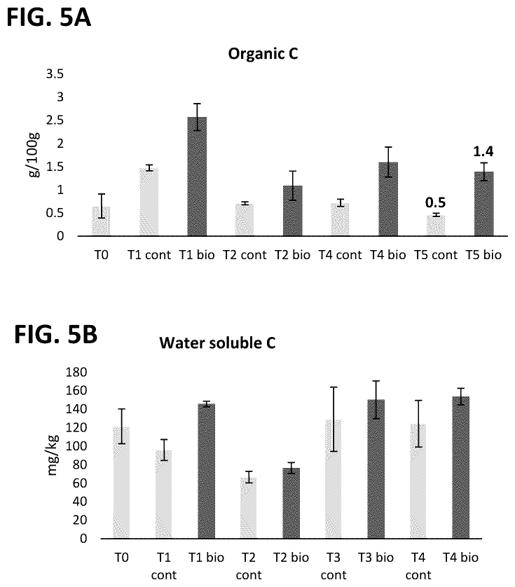

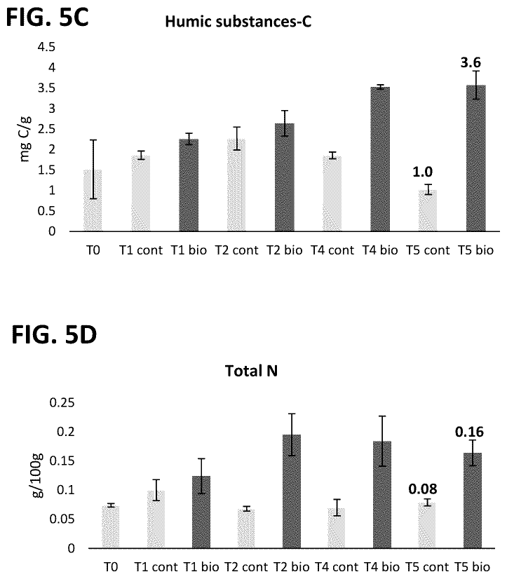

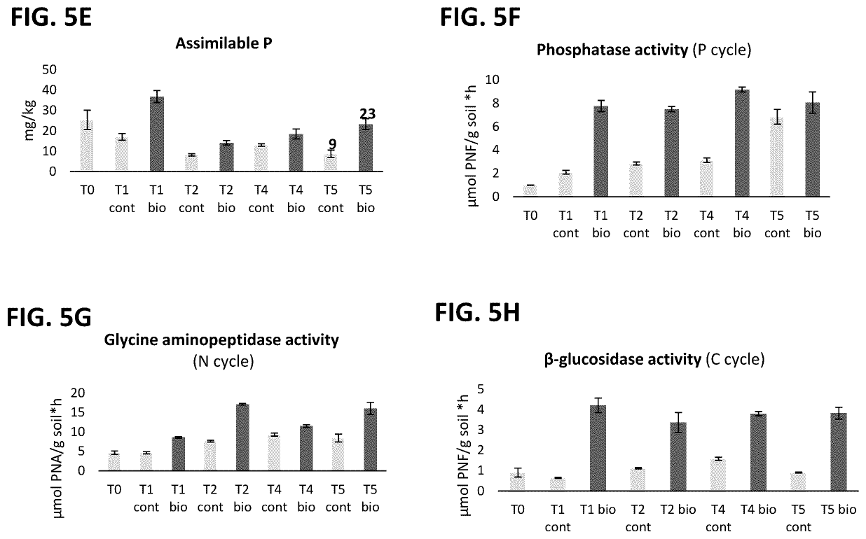

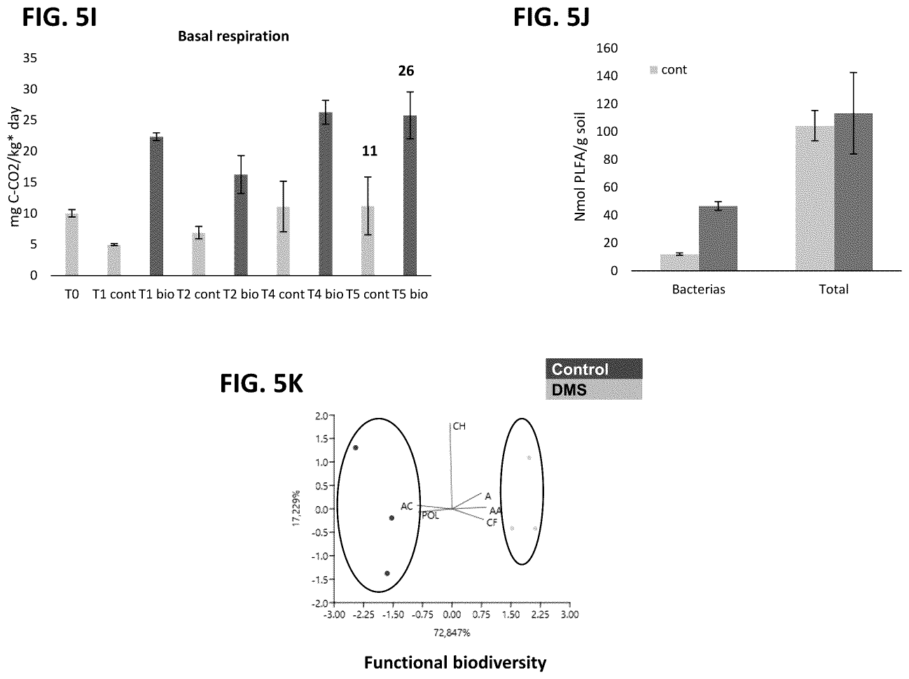

FIG. 4A-40 show the results for the soil of young trees, while FIG. 5A-5K show the results for the soil of adult trees. These results demonstrate that treated soil had higher organic carbon, water soluble carbon, water soluble polyphenols, humic substances, total nitrogen, water soluble nitrogen, nitrate, assimilable phosphorous, alkaline phosphatase activity, beta-glucosidase activity, urease activity, basal respiration, structural biodiversity, and functional biodiversity than control soil in one or both conditions at one or more time points after application of a microalgae composition of the present disclosure. These results demonstrate an increase in available nutrients (C, N, and P), a stimulation of microbial development, respiration, and hydrolase activities, and an increase in the size and diversity of the microbiome, with a preferential development of gram positive bacterial communities, especially Actinobacteria. These changes in soil parameters were surprisingly not reflected in a nutritional composition analysis of leaves in the host plants. In experiments performed on mandarin trees, young and old almond trees, and apricot trees, foliar analysis subsequent to fifth application of DMS did not establish, in general, differences between the treated and untreated trees in relation to the nutritional content of the leaf in terms of macronutrients, micronutrients, and other chemical elements and heavy metals. In lettuce, leaf nutrient composition was not significantly different between DMS+fertilizer and fertilizer-only control.

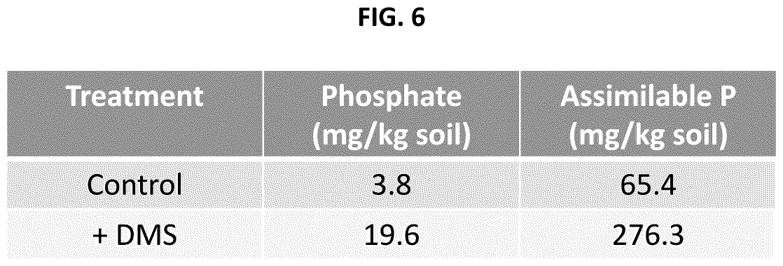

In a follow up study in lettuce, the positive changes in phosphate and assimilable phosphorous were confirmed in the soil of lettuce plants, following application of DMS (FIG. 6).

In further analyses of the soil of corn plants following application of 3 L/ha of 0.3% DMS at sowing, multiple differences were observed compared to control. The soil was evaluated to have experienced a beneficial modification of microbial populations, activation of P cycle, improvement of P mobilization, activation of K pathways, improvement of K solubilization, improvement of N cycling, improvement of microelements mobilization (increase in Fe assimilation and in transport of Mg and Cl), improvement in microbial phytohormones (increase in EPS, ACC deaminase, and heavy metal solubilization), adaption to stress at an early time point, and an increased effect of biocontrol agents against nematodes and insects at a later time point.

These results demonstrate an overall improvement in soil fitness and soil microbiome population, diversity, and activity following application of microalgae compositions of the disclosure.

### Example 5: Application of Microalgae Composition Provides Improved Drought Resistance and Recovery

Two tomato plants were subjected to drought conditions to determine the effect of DMS application on protection against drought and on a host plant's ability to recover from drought. The tomato plants were grown in identical conditions with 16 hours of light and 8 hours of darkness per day. The temperature during the day was 24-25° C., and at night, 20-22° C. The condition of the tomato plants was carefully documented over the course of 28 days with a photo time course. The schedule of irrigations and treatments is shown in Table 1.

TABLE 1
Schedule of irrigation and DMS treatments.

| Day | Treatment |
|---|---|
| 2 | Drought irrigation: 50 ml to each plant. Foliar treatment (spray) with DMS to the |
| | plant on the left with only water to the plant on the right. |
| 7 | Drought irrigation: 50 ml to each plant. Foliar treatment (spray) with DMS to the |
| | plant on the left with only water to the plant on the right. |
| 11 | Drought irrigation: 50 ml to each plant. |
| 14 | Drought irrigation: 50 ml to each plant. Foliar treatment (spray) with DMS to the |
| | plant on the left with only water to the plant on the right. |
| 17 | Irrigation: maximum retention capacity 500 ml to each plant. |
| 20 | Irrigation: maximum retention capacity 500 ml to each plant. Foliar treatment |
| | (spray) with DMS to the plant on the left with only water to the plant on the |
| | right. |
| 24 | Irrigation: maximum retention capacity 500 ml to each plant. |
| 27 | Irrigation: maximum retention capacity 500 ml to each plant. Foliar treatment |
| | (spray) with DMS to the plant on the left with only water to the plant on the right. |

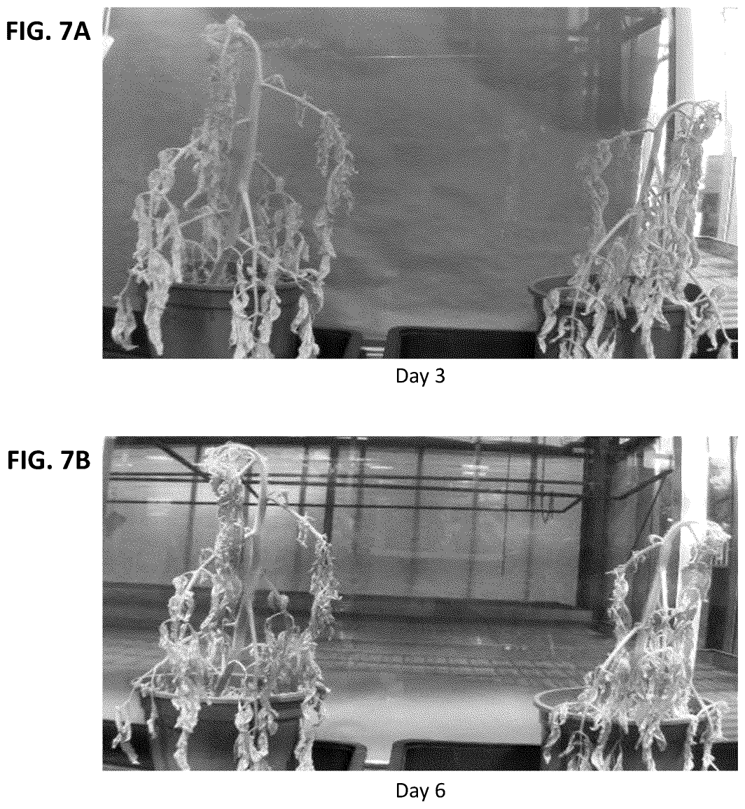

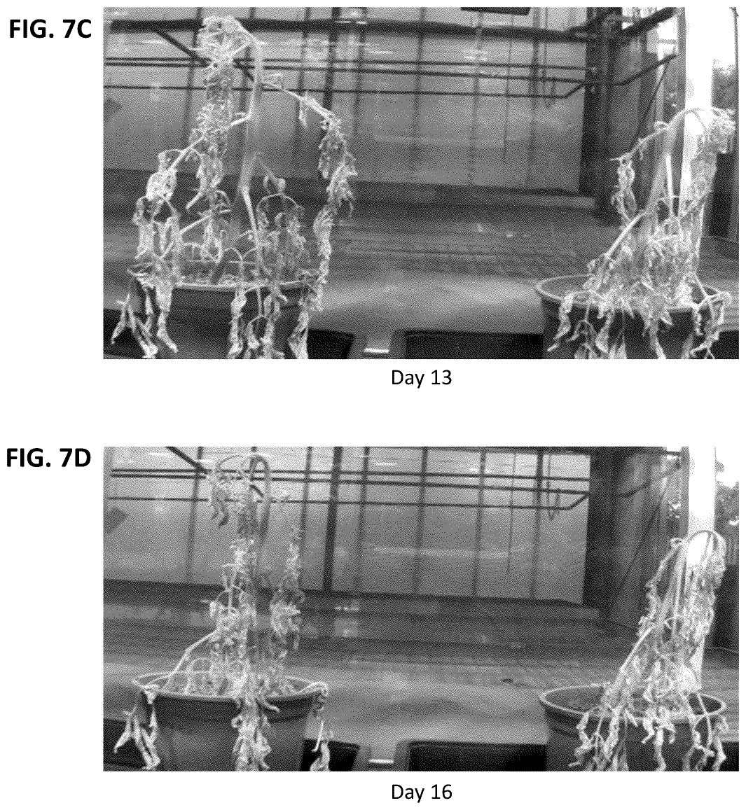

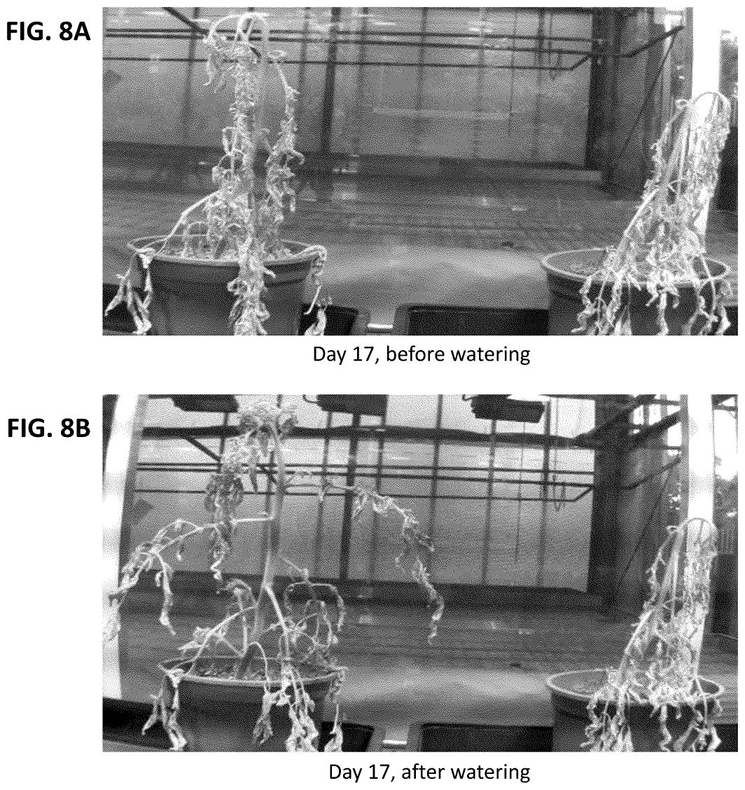

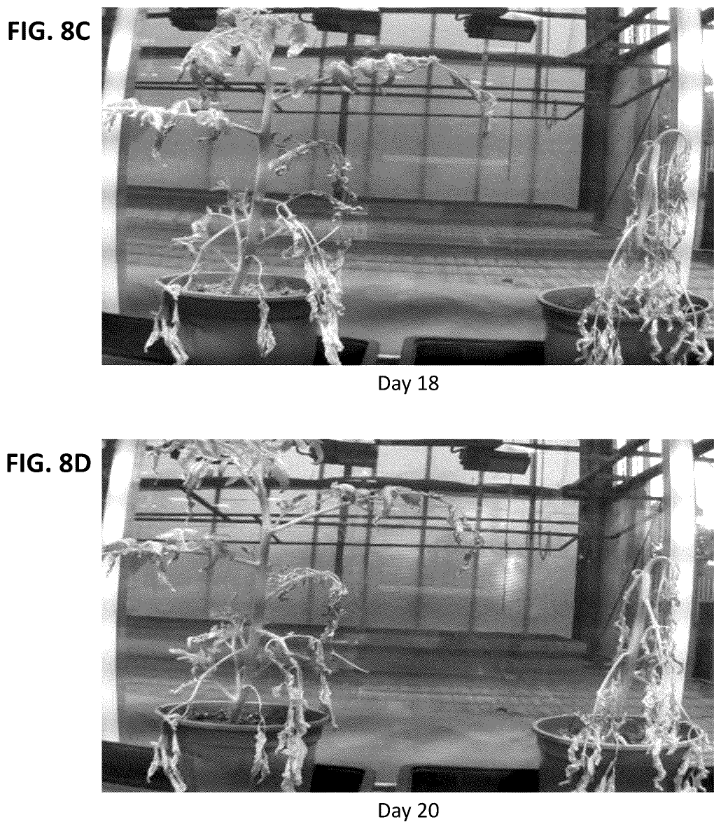

Foliar treatment (spraying) of DMS was carried out once a week on the treatment plant (plant on the left in FIG. 7A-7D, FIG. 8A-8E) while the control plant (plant on the right in FIG. 7A-7D, FIG. 8A-8E) was sprayed with only water as a control.

Irrigation was carried out twice a week under a dry regime from day 1 to day 17 with 50 ml (1/10 of the total retention capacity of the substrate in the pot). It was observed that the plants progressively suffered water stress, with the control plant being visibly more affected by the drought conditions. See FIG. 7A-7D. In particular, see FIG. 7C demonstrating the difference between the plants at Day 13. By Day 17, the plants appeared similarly wilted.

From the 17th day, watering was resumed with the maximum substrate retention capacity of 500 ml per pot twice a week. One foliar application per week of DMS on the treated plant and water on the control plant was continued. Within the span of a single day, faster recovery of the plant treated with DMS was observed compared to the untreated one. See FIG. 8B and FIG. 8C. FIG. 8C-8E provide additional images of the recovery after normal watering was resumed.

By day 28 of the experiment, the DMS-treated tomato plant was significantly larger and leafier than the untreated control, with remarkable new leaf growth. In addition, the majority of limbs and leaves that had suffered from drought stress on the DMS treated plant recovered once normal watering resumed. By contrast, the majority of branches and leaves on the untreated control plant had not recovered by day 28 and only limited new leaf growth was visible. Thus, even after 11 days of normal watering, the untreated control did not achieve a level of recovery similar to that of the treated one. See FIG. 8E.

### Example 6: Application of Microalgae Composition Improves Host Plant Resistance to Herbicidal Stress

Eggplant crops were grown in identical conditions with the recommended dose of fertilizer (“RDF”) and regular irrigation. The untreated control group had RDF alone. The treatment group had RDF with the addition of DMS, formulated as in Example 1, applied via two foliar applications. Broadleaf herbicide drift from neighboring rice fields exposed the plants in both groups to herbicidal stress.

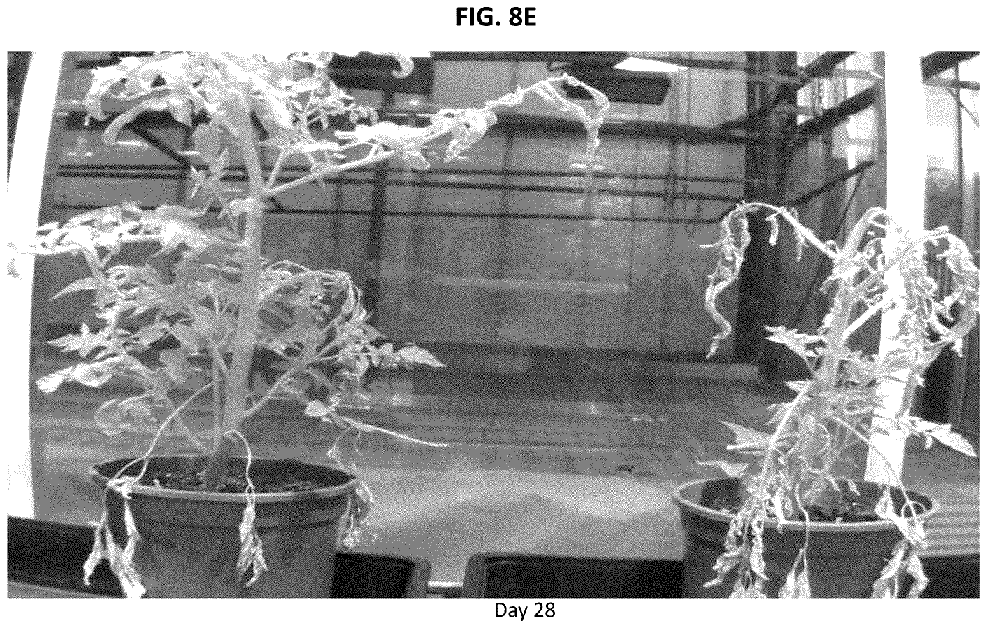

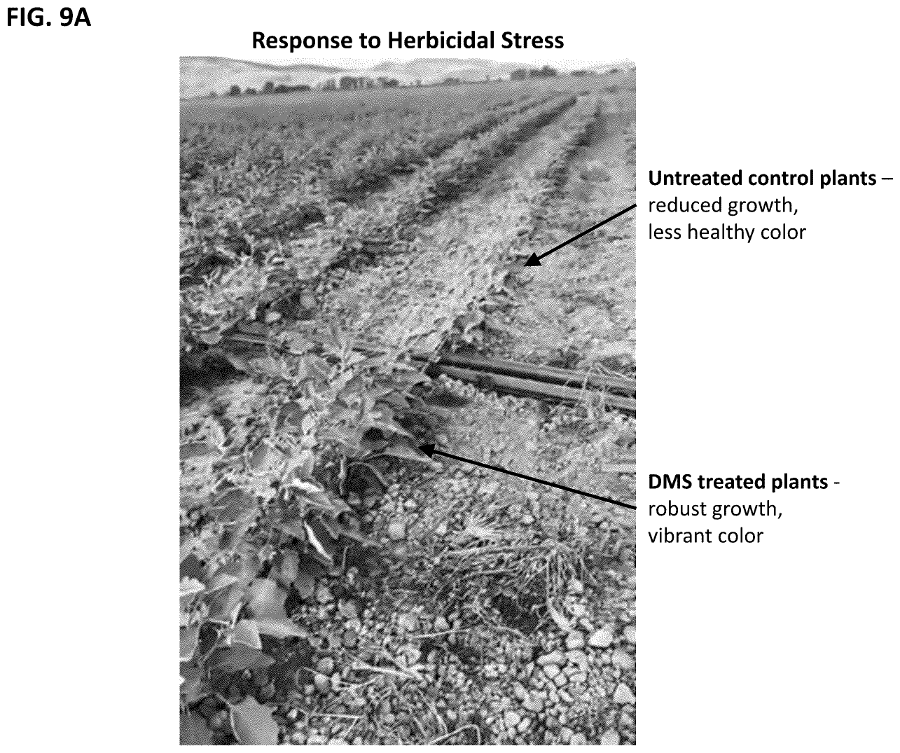

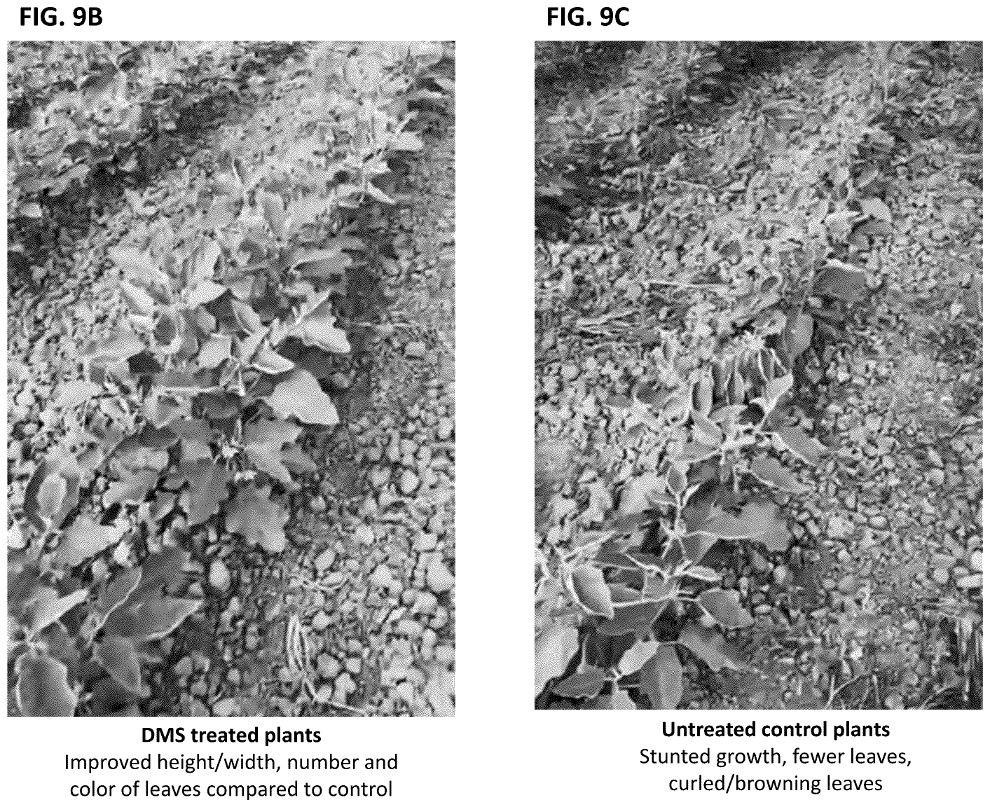

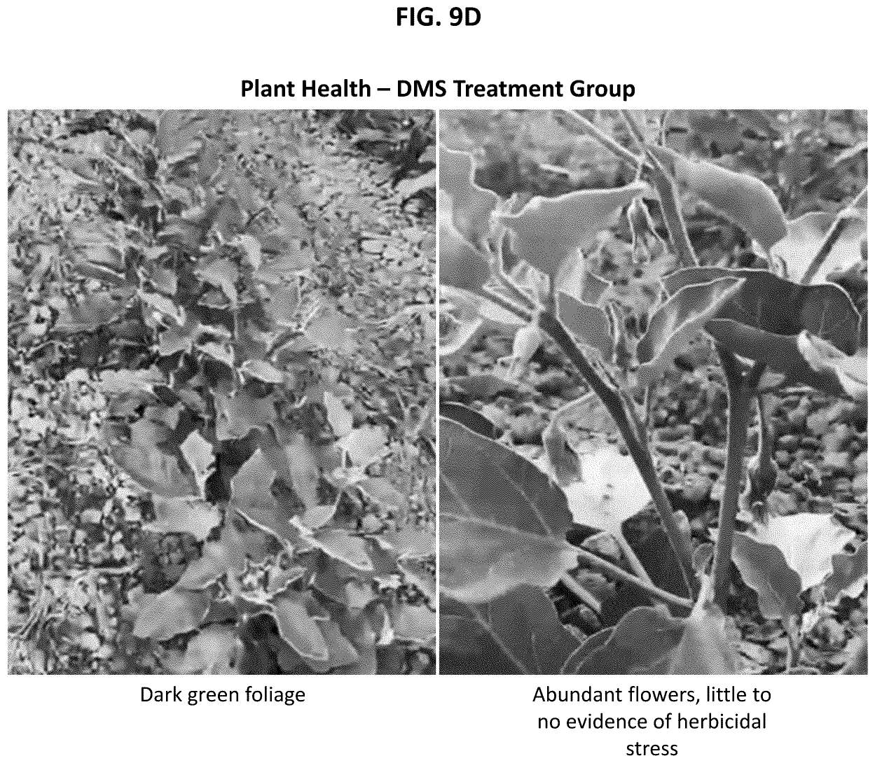

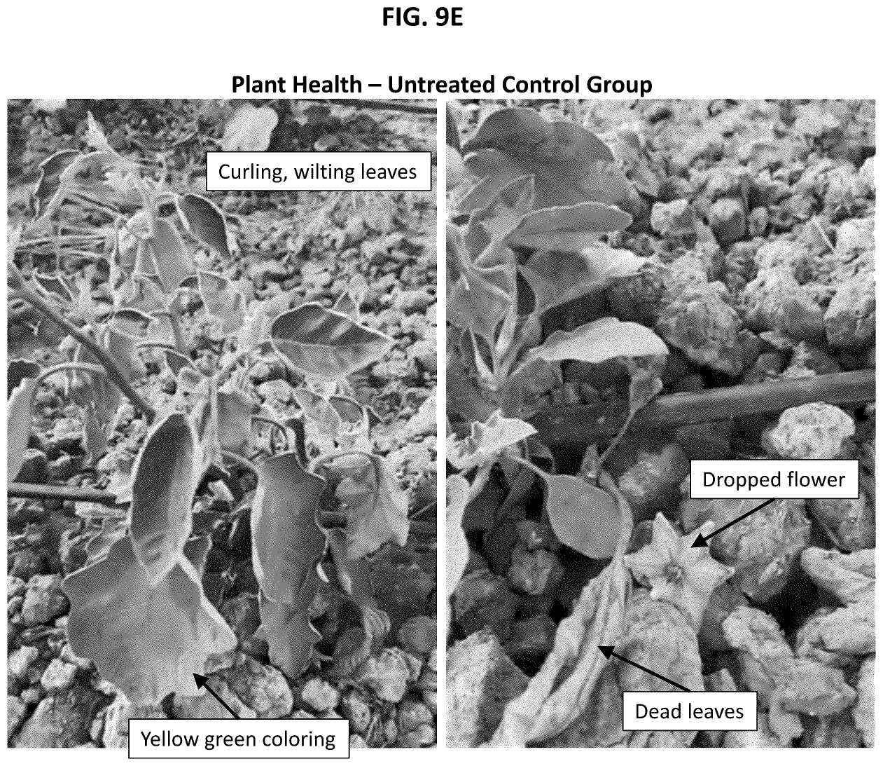

The plants that had been treated with DMS demonstrated improved resistance to and recovery from herbicidal stress compared to the untreated control plants (FIG. 9A). Spray damage was evident on the untreated eggplants: untreated plants exhibited stunted growth, wilting, yellowing, fewer leaves, curling leaves, dead leaves, and dropped flowers (FIG. 9C and FIG. 9E). By contrast, the DMS-treated eggplants maintained healthy leaf appearance and color, with robust growth, height, width, number of leaves, number of flowers, and vibrant green color (FIG. 9B and FIG. 9D).

### Example 7: Genes Upregulated in Host Plant Response to Abiotic Stress after Application of Illustrative Microalgae Composition

As in Example 3, gene upregulation in tomato. Arabidopsis, and canola plants was measured following DMS application to identify upregulated genes involved in abiotic stress and drought resistance. The analysis determined that PYL, PP2C, SnRK2, AREB, ABF, and MAPK gene expression upregulation was identified in some host plants following application of DMS (FIG. 10A-10B). These genes are implicated in host plant response to abiotic stress and drought stress resistance.

### Example 8: Microalgae Composition Application Improves Soil Microbial Diversity and Nutrient Attributes

### Materials and Methods

DMS was applied at a rate of 3 L/ha to the soil of corn crops. Samples for untreated and treated soil were taken before application (TO time point) and after application (T1 and T2 time points). DMS was applied at TO after sampling: T1 was 14 days after T0; and T2 was 35 days after TO. Soil samples were collected in triplicate in sterile tubes at a depth of 2-6 inches. Samples were collected as a composite of several cores in each treatment block, in order to ensure accurate representation of the area. The samples were analyzed for markers of microbial activity, microbial nutrient solubilization, nutrient cycling, stress adaptation, and biocontrol activity. Statistically significant changes having a significance value of between 0.1 and 0.3 are marked with a single asterisk in the figures discussed in the results below. A major change observed in more than one location and/or time point, but with a statistical significance greater than 0.3, is indicated with a box with a dashed line around the result.

### Results

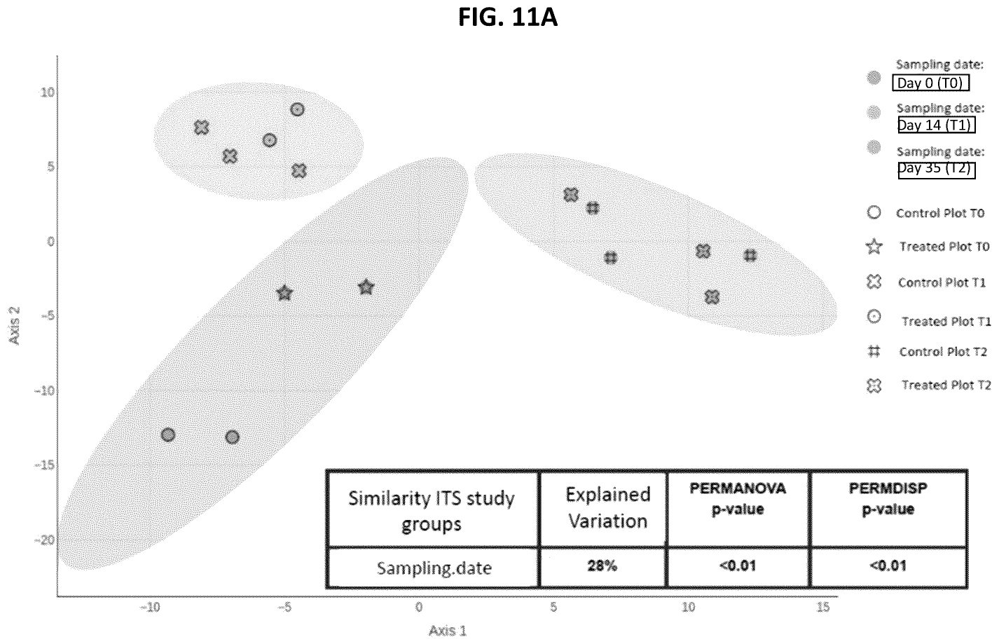

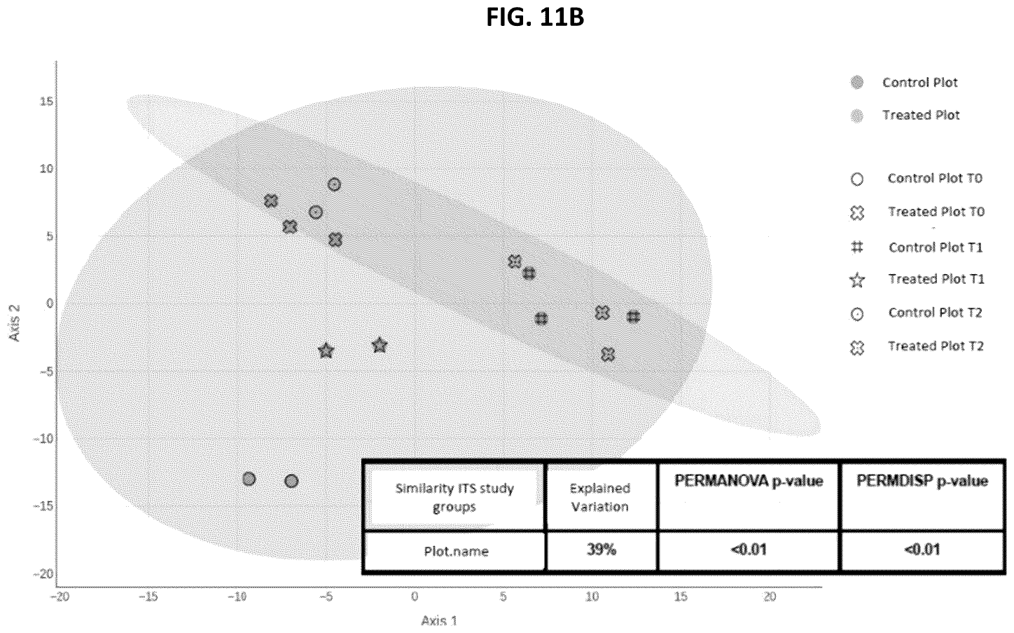

Microbial beta-diversity. Use of the product was able to significantly modulate the microbiome communities present in the soil over time, to the plant's benefit. FIG. 11A shows three distinct populations were revealed as a function of sampling date, while FIG. 11B shows the identification of two distinct populations, differing by treatment condition. Permanova analysis in both cases yielded p<0.01.

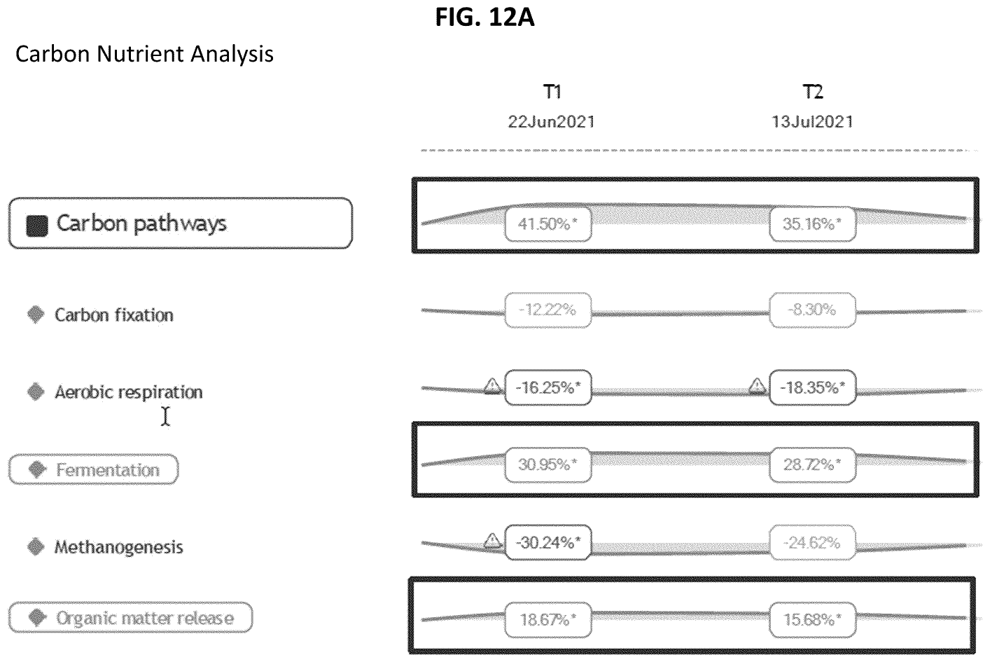

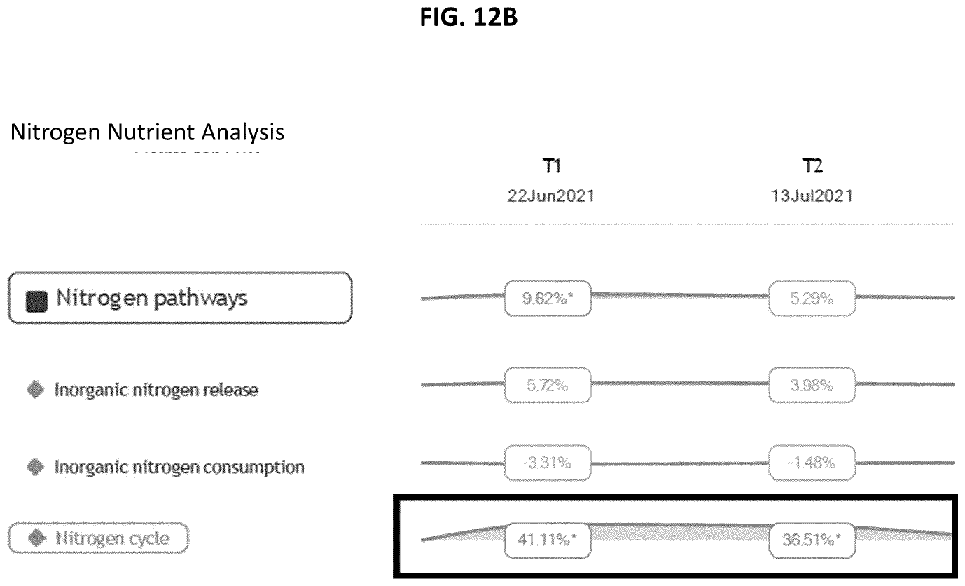

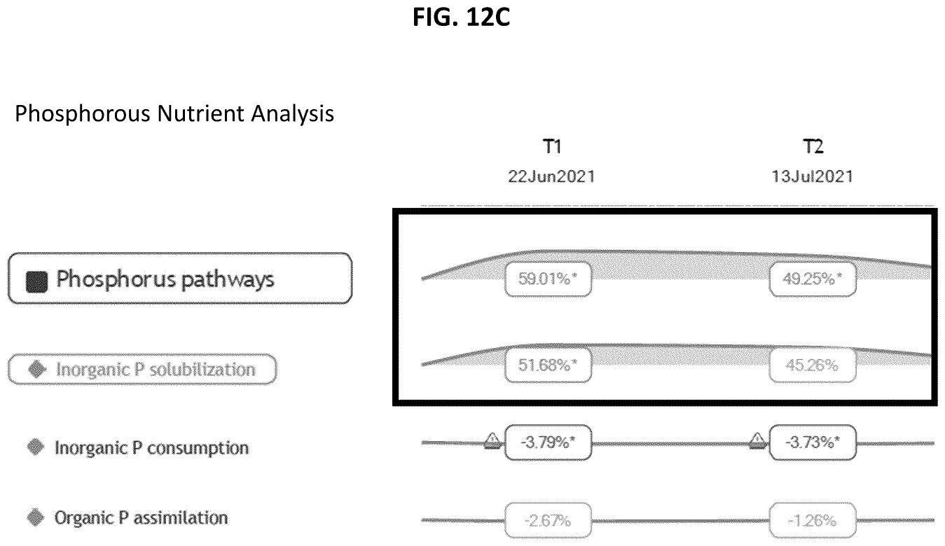

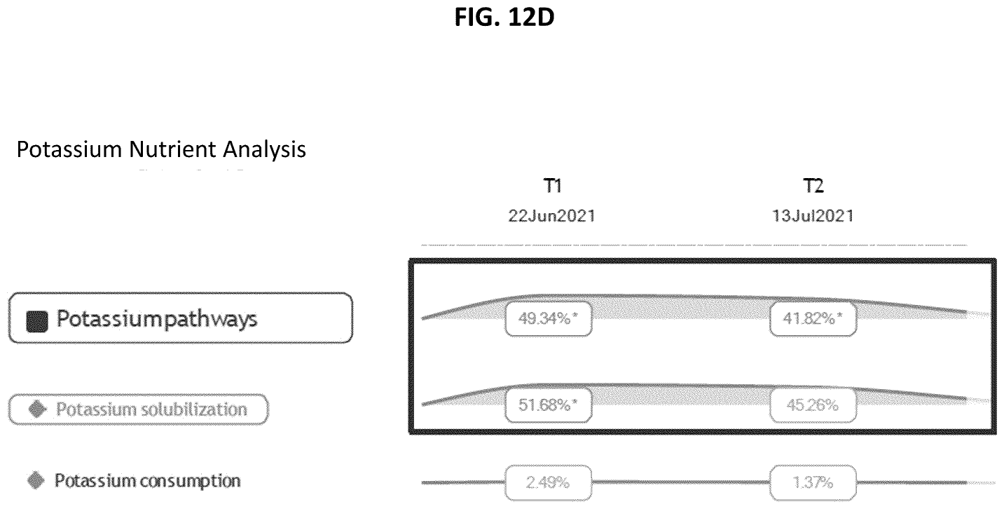

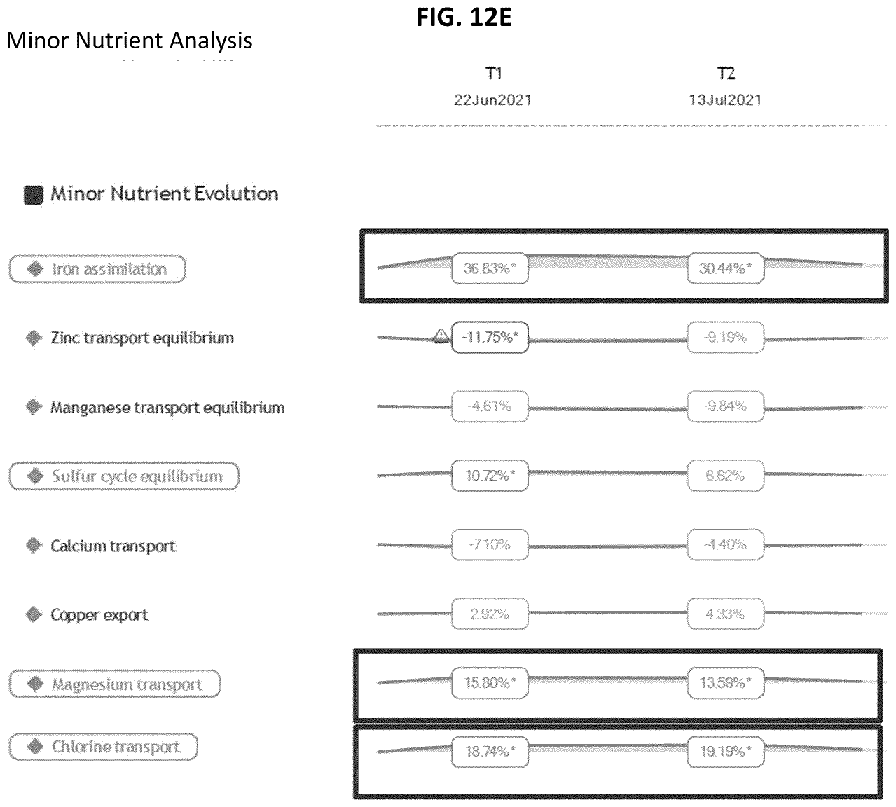

Nutrient analysis. The potential for nutrient cycling of bacterial communities associated with all samples across the treatment blocks were analyzed using the abundance of each bacteria and the known metabolic enzyme compositions of their genomes.
Carbon: This analysis revealed elevated carbon pathways, fermentation, and organic matter release in the treated condition compared to the control at both T1 and T2. See FIG. 12A.
Nitrogen: nitrogen pathways and nitrogen cycling were elevated in the treated condition compared to the control. Nitrogen cycling in particular was statistically significantly elevated at both T1 and T2, demonstrating that the improvement was maintained over time. See FIG. 12B.
Phosphorous: Phosphorous pathways and inorganic P solubilization were elevated in the treated condition at both T1 and T2, demonstrating enhanced phosphorous mobilization maintained over time. See FIG. 12C.
Potassium: Potassium pathways and potassium solubilization were elevated in the treated condition at both T1 and T2 compared to the control. See FIG. 12D.
Minor nutrients: Iron assimilation, magnesium transport, and chlorine transport were elevated at both T1 and T2 in the treated condition compared to the control. See FIG. 12E.

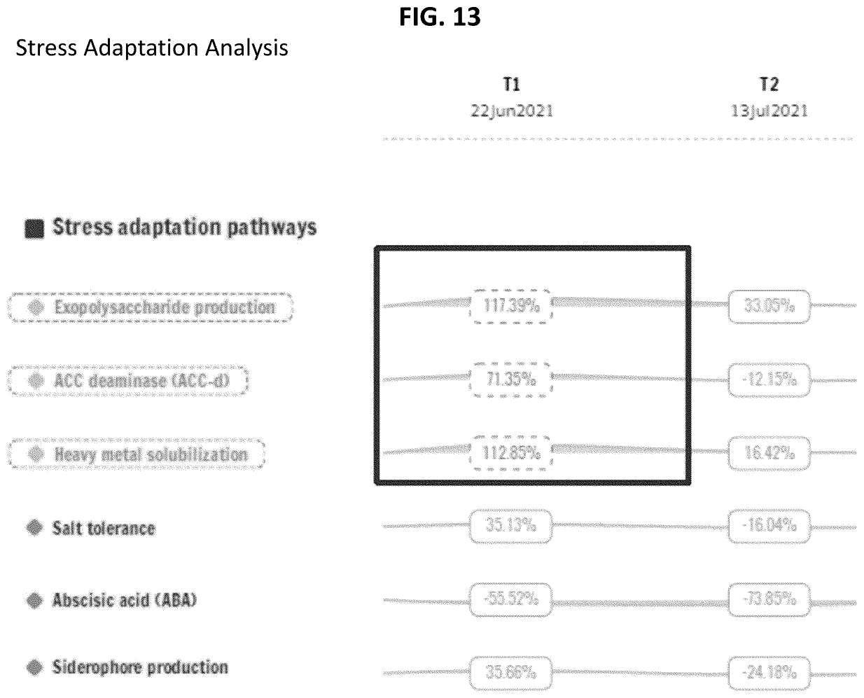

Stress adaptation pathways. Indicators of adaptation to stress were active at T1 in the treated condition compared to the control. In particular, exopolysaccharide production, ACC deaminase (ACC-d) activity, and heavy metal solubilization were all elevated in the treated condition at T1. See FIG. 13.

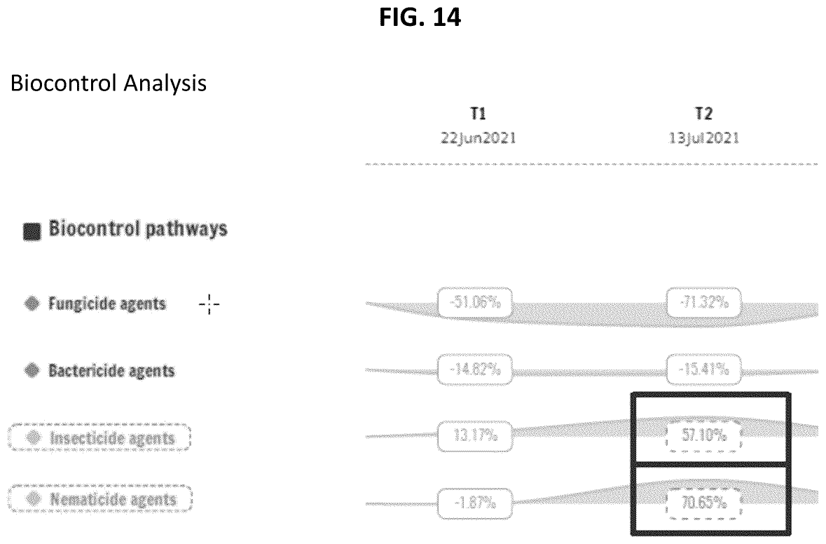

Biocontrol pathways. The presence of biocontrol agents were analyzed in all samples across the different treatment blocks. An increased number of nematicidal and insecticidal biocontrol agents were observed in the treated soil at T2, as compared to the control soil. See FIG. 14.

The above results demonstrate an overall improvement in soil fitness, microbial diversity, nutrient solubilization, and nutrient cycling in the soil of com crops following application of an illustrative microalgae composition of the disclosure.

### Example 9: Illustrative Microalgae Composition Decreases NPK Reliance in Field Trial in Corn

### Materials & Methods

To investigate the effect of microalgae compositions in reducing reliance on NPK, a field trial was conducted in corn comparing a control with full fertilizer application and a treated condition with at least 20% reduced NPK fertilizer application, along with DMS application. DMS was applied as a foliar spray at a rate of 3 L/ha in the treated plot. The details of fertilizer and sowing times and amounts are shown in Table 2 for the control plot and Table 3 for the DMS-treated plot.

TABLE 2
Fertilizer and sowing details for control plot.

| Application | Quantity | Date from Sowing |
|---|---|---|
| Background Fertilization | 450 kg/ha of 13-11-21 NPK | Day −2 |
| Starter Fertilization | 150 kg/ha of 21-17-0 NPK | Day 0 |
| Sowing | 95,000 plants/ha | Day 0 |
| Herbicide | 4 L LUMAX ®/ha + 100 mL Ninja ®/ha | Day 0 |
| Coverage fertilization | 500 kg/ha of 40-0-0 NPK | Day 31 |

TABLE 3
Fertilizer and sowing details for DMS-treated plot.

| Application | Quantity | Date from Sowing |
|---|---|---|
| Background Fertilization | 350 kg/ha of 13-11-21 NPK (−22%) | Day −2 |
| Starter Fertilization | 120 kg/ha of 21-17-0 NPK (−20%) | Day 0 |
| Sowing | 95,000 plants/ha | Day 0 |
| Herbicide | 4 L LUMAX ®/ha + 100 mL Ninja ®/ha | Day 0 |
| Coverage fertilization | 400 kg/ha of 40-0-0 NPK (−20%) | Day 31 |
| Microalgae composition | 3 L/ha of DMS on 4/6 leaves | Day 17 |

Control and treated plots were tested for average corn yield (kg/ha), average com humidity (%), fertilization costs (€/ha), and projected return on investment (€/ha).

### Results

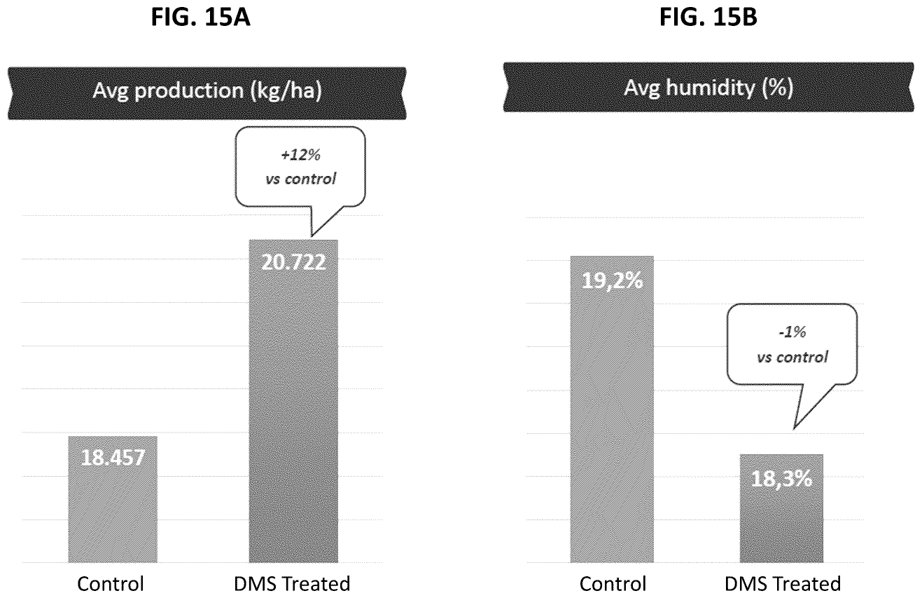

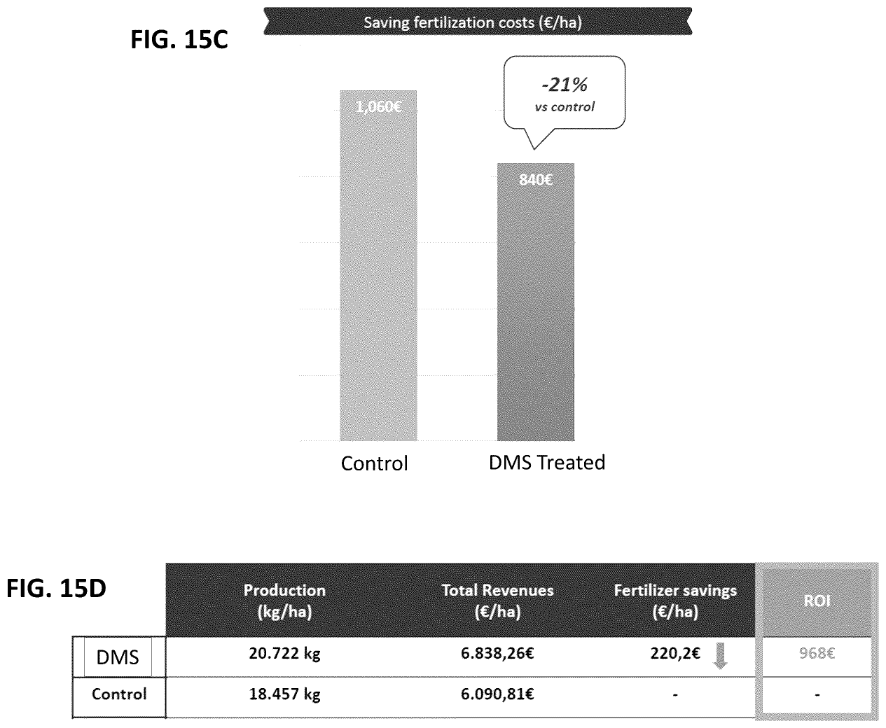

The average production in terms of kg of corn/ha was 12% higher for the DMS-treated condition, in spite of 20% lower NPK fertilizer application compared to control (FIG. 15A). Average humidity was 1% lower compared to control (FIG. 15B). Fertilizer costs were 21% lower compared to the control, even including the cost of the DMS treatment (FIG. 15C). Based on the yield and the expected revenues, along with the savings in fertilizer cost, the projected return on investment was 968€/ha for the DMS-treated condition with reduced fertilizer compared to the control condition (FIG. 15D).

### Example 10: Illustrative Microalgae Composition Decreases NPK Reliance in Field Trial in Barley

### Materials & Methods

A field trial was conducted in barley comparing a control plot with full fertilizer application to four DMS-treated conditions with 100%, 90%, 80%, or 70% NPK fertilizer application. The details of the different treatment conditions are shown in Table 4 below.

TABLE 4
Treatment conditions.

| Treatment Group | Treatment Condition |
|---|---|
| T1 (Control) | 100% N fertilizer |
| T2 | 100% N fertilizer + 1 L/ha DMS |
| T3 | 90% N fertilizer + 1 L/ha DMS |
| T4 | 80% N fertilizer + 1 L/ha DMS |
| T5 | 70% N fertilizer + 1 L/ha DMS |

In all treatment conditions, seeds were sowed at a density of 350 germinable seeds/m2. Background NPK fertilizer (Dec. 24, 2012) was applied on the day of sowing. Emergence was observed 9 days following sowing. Herbicide was applied 35 days after sowing. DMS was applied as a foliar spray 45 days after sowing at a rate of 1 L/ha in each of T2-T5 conditions. Coverage fertilization was carried out on day 78 after sowing. Fungicide was applied on day 92. Spiking of the barley plant was observed around 106 days, or 3.5 months after sowing. Maturation occurred at approximately day 153, or about 5 months after sowing. Barley was harvested at 223 days, about 7.5 months, after sowing.

Control and treated plots were tested for yield, mass, percent protein, Normalized Difference Vegetation Index, and projected return on investment.

### Results

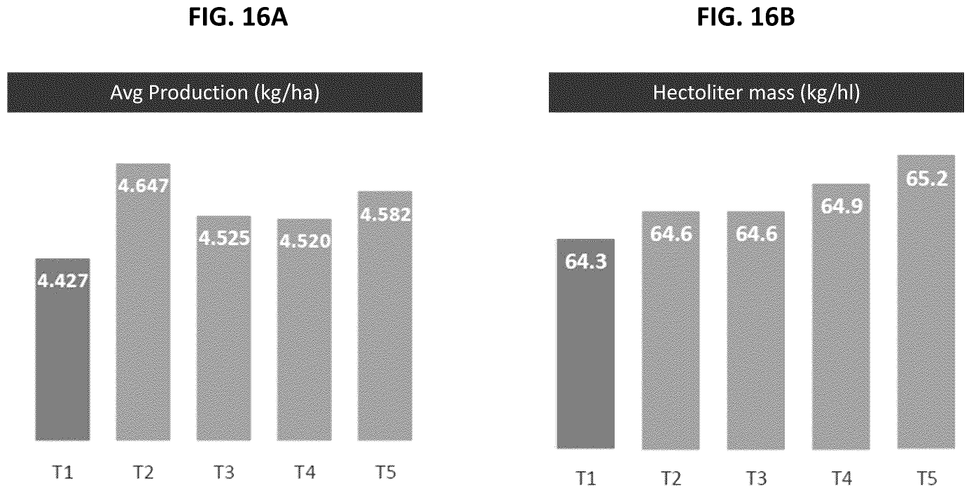

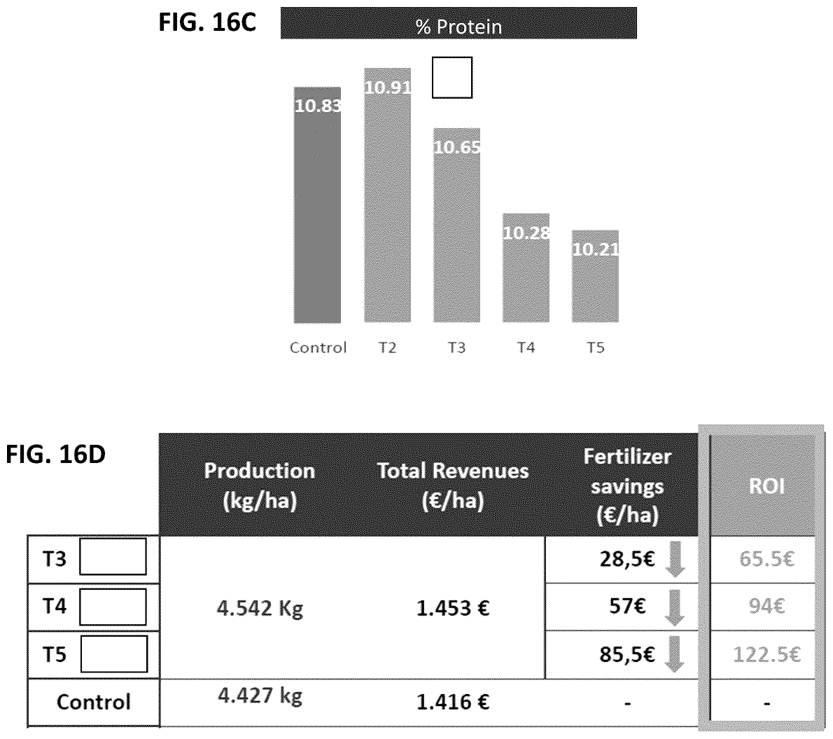

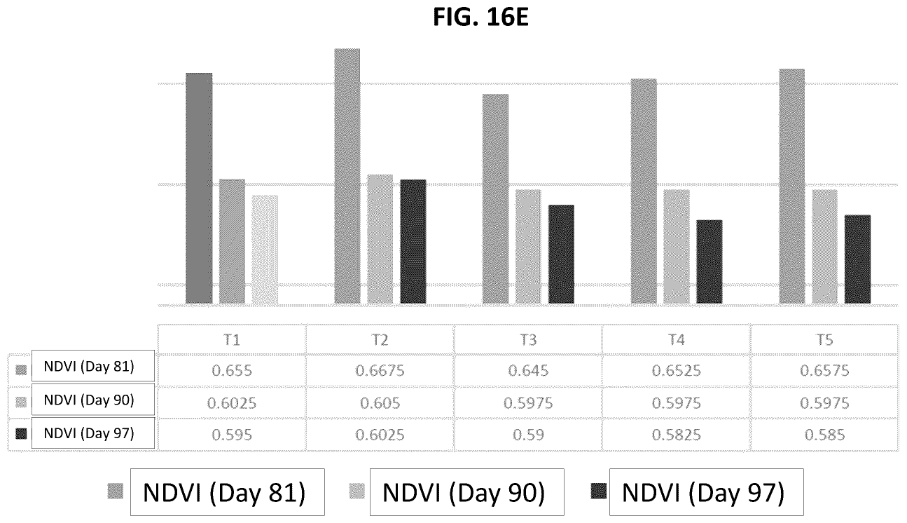

The average yield in terms of kg/ha of barley was 2.5% higher averaged across DMS-treated groups, in spite of 10-30% lower N fertilizer application in treatments T3-T5 (FIG. 16A). Average mass per hectoliter volume (100 L) was within variance for all treatment conditions (FIG. 16B). Percent protein was slightly lower in conditions with decreased fertilizer, but within about 0.6% for the condition with the least fertilizer application (FIG. 16C). Based on the yield and the expected revenues, along with the savings in fertilizer cost, the projected return on investment was 65.5 €/ha for T3: 94 €/ha for T4; and 122.5 €/ha for T5 compared to the control condition (FIG. 16D). NDVI measurements were taken at 81 days, 90 days, and 97 days after sowing (see bars from left to right and table entries from top to bottom in FIG. 16E). A Tukey's test analysis of the NDVI measurements demonstrated no statistically significant differences between all five test conditions.

These results demonstrate slightly higher yield and comparability in other metrics even with significantly reduced (up to 30% lower) N fertilizer following application of DMS in a barley field trial.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

### Numbered Embodiments of the Invention

Notwithstanding the appended claims, the disclosure sets forth the following numbered embodiments:
1. A method for improving host plant nutrient utilization, the method comprising the step of:
   a) applying a microalgae-based formulation to the host plant.
2. The method of embodiment 1, wherein the method upregulates expression of genes involved in nitrogen metabolism.
3. The method of any one of embodiments 1-2, wherein the method upregulates expression of nitrate reductase, glutamate dehydrogenase, and/or glutamate synthase.
4. The method of any one of embodiments 1-3, wherein the method upregulates expression of genes involved in amino acid metabolism.
5. The method of any one of embodiments 1-4, wherein the method upregulates expression of AGT, ALAAT, ASN, and/or GDH genes.
6. The method of any one of embodiments 1-5, wherein the formulation is applied to the host plant, a plant part of the host plant, or the environs of the host plant.
7. The method of any one of embodiments 1-6, wherein the method decreases the amount of exogenous nitrogen required by the host plant.
8. The method of any one of embodiments 1-7, wherein the method decreases the amount of exogenous nitrogen required by the host plant by at least 25% without negatively impacting host plant yield.
9. The method of any one of embodiments 1-8, wherein the method produces the same or higher value of a growing parameter, production parameter, or biostimulant parameter compared to a control plant without the microalgae-based formulation, with less exogenous nitrogen application than the control plant.
10. A method for improving soil fitness, the method comprising the step of:
   a) applying a microalgae-based formulation to the soil.
11. The method of embodiment 10, wherein the method enhances nutrient availability in the soil.
12. The method of any one of embodiments 10-11, wherein the method increases the soil content of a nutrient selected from the list consisting of: phosphate, assimilable phosphorous, organic carbon, water soluble carbon, humic substances, total nitrogen, water soluble nitrogen, nitrate, and water soluble polyphenols.
13. The method of any one of embodiments 10-12, wherein the method changes the nutrient composition of the soil, but does not change the nutrient composition of the leaves of a host plant planted therein.
14. A method for stimulating rhizospheric growth and activity within a soil, the method comprising the step of:
   a) applying a microalgae-based formulation to the soil.
15. The method of embodiment 14, wherein the method stimulates microbial development and/or respiration.
16. The method of any one of embodiments 14-15, wherein the method increases the level of alkaline phosphatase activity, beta-glucosidase activity, urease activity, basal respiration, glycine aminopeptidase activity, and/or hydrolase activity in the soil.
17. The method of any one of embodiments 14-16, wherein the method increases the biodiversity of the soil microbiome.
18. The method of any one of embodiments 14-17, wherein the method increases the concentration of microbes per gram of soil.
19. The method of any one of embodiments 14-18, wherein the method increases the concentration of gram positive bacteria, gram negative bacteria, and/or fungi in the soil.
20. The method of any one of embodiments 14-19, wherein the method preferentially increases the concentration of gram positive bacteria in the soil.
21. The method of any one of embodiments 14-20, wherein the method preferentially increases the concentration of actinobacteria in the soil.
22. The method of any one of embodiments 1-21, wherein the formulation comprises multiple species of microalgae.
23. The method of any one of embodiments 1-22, wherein the formulation comprises microalgae from a phylum selected from the list consisting of: Chlorophyta, Cryptophyta, Cyanophyta, Euglenophyta, Heterokontophyta, or Rhodophyta.
24. The method of any one of embodiments 1-23, wherein the formulation comprises microalgae from a genus selected from the list consisting of: Chlorella, Scenedesmus, Nannochloropsis, Muriellopsis, Isochrysis, Tisochrysis, Desmodesmus, Haematococcus, Arthrospira, and Anabaena.
25. The method of any one of embodiments 1-24, wherein the formulation comprises whole-cell microalgae powder.
26. The method of any one of embodiments 1-25, wherein the formulation comprises 0.1-50 g/L of whole-cell microalgae powder.
27. The method of any one of embodiments 1-26, wherein the formulation comprises 0.8-20 g/L of whole-cell microalgae powder.
28. The method of any one of embodiments 1-27, wherein the formulation comprises digested microalgae solution (“DMS”).
29. The method of any one of embodiments 1-28, wherein the formulation comprises 0.3-0.5% v/v DMS.
30. The method of any one of embodiments 1-29, wherein the formulation comprises 5-15% dry matter of microalgae.
31. The method of any one of embodiments 1-30, wherein the formulation comprises 5-15% dry matter of microalgae and is diluted to 0.3-0.5% v/v in water prior to application.
32. The method of any one of embodiments 1-31, wherein the formulation is a liquid, and wherein the formulation is applied at a rate of 0.5-20 L/ha.
33. The method of any one of embodiments 1-32, wherein the formulation is a liquid, and wherein the formulation is applied at a rate of 1-10 L/ha.
34. The method of any one of embodiments 1-33, wherein the formulation is a granule formulation, and wherein the formulation is applied at a rate of 1-20 kg/ha.
35. The method of any one of embodiments 1-34, wherein the formulation is a granule formulation, and wherein the formulation is applied at a rate of 5-15 kg/ha.
36. A method for improving host plant resistance to abiotic stress, the method comprising the step of:
   a) applying a microalgae-based formulation to the host plant.
37. A method for improving host plant recovery from abiotic stress, the method comprising the step of:
   a) applying a microalgae-based formulation to the host plant.
38. The method of embodiment 36 or 37, wherein the abiotic stress is water stress, temperature stress, sun stress, salinity stress, wind stress, herbicidal stress, or heavy metal stress.
39. The method of any one of embodiments 36-38, wherein the abiotic stress is drought, heat, cold, excess salinity, herbicide exposure, strong winds, heavy metals, flooding, or excessive sunlight.
40. The method of any one of embodiments 36-39, wherein the abiotic stress is water stress.
41. The method of any one of embodiments 36-39, wherein the abiotic stress is drought.
42. The method of any one of embodiments 36-39, wherein the abiotic stress is temperature stress.
43. The method of any one of embodiments 36-39, wherein the abiotic stress is herbicidal stress.
44. The method of any one of embodiments 36-39, wherein the abiotic stress is salinity stress.
45. A method for improving a host plant's resistance to and/or recovery from abiotic stress, the method comprising the step of:
   a) applying a microalgae-based formulation to the host plant,
      wherein the abiotic stress is drought.
46. The method of any one of embodiments 36-45, wherein the method decreases the damage to a growing parameter, production parameter, or biostimulant parameter of the host plant incurred by exposure to the abiotic stress.
47. The method of any one of embodiments 36-46, wherein the method decreases loss of yield as a result of exposure to the abiotic stress.
48. The method of any one of embodiments 36-47, wherein the method improves recovery from the damage to a growing parameter, production parameter, or biostimulant parameter of the host plant incurred by exposure to the abiotic stress.
49. The method of any one of embodiments 36-48, wherein the method decreases the amount of recovery time required by the host plant following exposure to the abiotic stress.
50. The method of any one of embodiments 36-49, wherein the method upregulates expression of genes involved in resistance to abiotic stress and/or drought resistance.
51. The method of any one of embodiments 36-50, wherein the method upregulates expression of a gene encoding an ABA receptor, a protein phosphatase 2C, an SNF1-related protein kinase 2, an ABRE-binding protein, an ABRE-binding factor, and/or a mitogen-activated protein kinase.
52. The method of any one of embodiments 36-51, wherein the method upregulates expression of a PYL, PP2C, SnRK2, AREB, ABF, and/or MAPK gene.
53. The method of any one of embodiments 36-52, wherein the formulation comprises multiple species of microalgae.
54. The method of any one of embodiments 36-53, wherein the formulation comprises microalgae from a phylum selected from the list consisting of: Chlorophyta, Cryptophyta, Cyanophyta, Euglenophyta, Heterokontophyta, or Rhodophyta.
55. The method of any one of embodiments 36-54, wherein the formulation comprises microalgae from a genus selected from the list consisting of: Chlorella, Scenedesmus, Nannochloropsis, Muriellopsis, Isochrysis, Tisochrysis, Desmodesmus, Haematococcus, Arthrospira, and Anabaena.
56. The method of any one of embodiments 36-55, wherein the formulation comprises whole-cell microalgae powder.
57. The method of any one of embodiments 36-56, wherein the formulation comprises 0.1-50 g/L of whole-cell microalgae powder.
58. The method of any one of embodiments 36-57, wherein the formulation comprises 0.8-20 g/L of whole-cell microalgae powder.
59. The method of any one of embodiments 36-58, wherein the formulation comprises digested microalgae solution (“DMS”).
60. The method of any one of embodiments 36-59, wherein the formulation comprises 0.3-0.5% v/v DMS.
61. The method of any one of embodiments 36-60, wherein the formulation comprises 5-15% dry matter of microalgae.
62. The method of any one of embodiments 36-61, wherein the formulation comprises 5-15% dry matter of microalgae and is diluted to 0.3-0.5% v/v in water prior to application.
63. The method of any one of embodiments 36-62, wherein the formulation is a liquid, and wherein the formulation is applied at a rate of 0.5-20 L/ha.
64. The method of any one of embodiments 36-63, wherein the formulation is a liquid, and wherein the formulation is applied at a rate of 1-10 L/ha.
65. The method of any one of embodiments 36-64, wherein the formulation is a granule formulation, and wherein the formulation is applied at a rate of 1-20 kg/ha.
66. The method of any one of embodiments 36-65, wherein the formulation is a granule formulation, and wherein the formulation is applied at a rate of 5-15 kg/ha.

1. A method for improving host plant nutrient utilization, the method comprising the step of:
a) applying a microalgae-based formulation to the host plant.

2. The method of claim 1, wherein the method upregulates expression of genes involved in nitrogen metabolism, optionally wherein the method upregulates expression of nitrate reductase, glutamate dehydrogenase, and/or glutamate synthase.

3. The method of claim 1, wherein the method upregulates expression of genes involved in amino acid metabolism, optionally wherein the method upregulates expression of AGT, ALAAT, ASN, and/or GDH genes.

4. The method of claim 1, wherein the formulation is applied to the host plant, a plant part of the host plant, or the environs of the host plant.

5. The method of claim 1, wherein the method decreases the amount of exogenous nitrogen, phosphorous, and/or potassium required by the host plant, optionally wherein the method decreases the amount of exogenous nitrogen required by the host plant by at least 25% without negatively impacting host plant yield.

6. The method of claim 1, wherein the method produces the same or higher value of a growing parameter, production parameter, or biostimulant parameter compared to a control plant without the microalgae-based formulation, with less exogenous nitrogen application than the control plant.

7. A method for improving soil fitness, the method comprising the step of:
a) applying a microalgae-based formulation to the soil.

8. The method of claim 7, wherein the method enhances nutrient availability in the soil.

9. The method of claim 7, wherein the method increases the soil content of a nutrient selected from the list consisting of: phosphate, assimilable phosphorous, organic carbon, water soluble carbon, humic substances, total nitrogen, water soluble nitrogen, nitrate, and water soluble polyphenols.

10. The method of claim 7, wherein the method changes the nutrient composition of the soil, but does not change the nutrient composition of the leaves of a host plant planted therein.

11. A method for stimulating rhizospheric growth and activity within a soil, the method comprising the step of:
a) applying a microalgae-based formulation to the soil.

12. The method of claim 11, wherein the method stimulates microbial development and/or respiration.

13. The method of claim 11, wherein the method increases the level of alkaline phosphatase activity, beta-glucosidase activity, urease activity, basal respiration, glycine aminopeptidase activity, and/or hydrolase activity in the soil.

14. The method of claim 11, wherein the method increases the biodiversity of the soil microbiome.

15. The method of claim 11, wherein the method increases the concentration of microbes per gram of soil, optionally wherein the method increases the concentration of gram positive bacteria, gram negative bacteria, and/or fungi in the soil.

16. The method of claim 11, wherein the method preferentially increases the concentration of gram positive bacteria in the soil, optionally wherein the method preferentially increases the concentration of actinobacteria in the soil.

17. The method of any one of claims 1, 7, and 11, wherein the formulation comprises multiple species of microalgae.

18. The method of any one of claims 1, 7, and 11, wherein the formulation comprises microalgae from a phylum selected from the list consisting of: Chlorophyta, Cryptophyta, Cyanophyta, Euglenophyta, Heterokontophyta, or Rhodophyta.

19. The method of any one of claims 1, 7, and 11, wherein the formulation comprises microalgae from a genus selected from the list consisting of: Chlorella, Scenedesmus, Nannochloropsis, Muriellopsis, Isochrysis, Tisochrysis, Desmodesmus, Haematococcus, Arthrospira, and Anabaena.

20. The method of any one of claims 1, 7, and 11, wherein the formulation comprises whole-cell microalgae powder, optionally wherein the formulation comprises 0.1-50 g/L or 0.8-20 g/L of whole-cell microalgae powder.

21. The method of any one of claims 1, 7, and 11, wherein the formulation comprises digested microalgae solution (“DMS”).

22. The method of any one of claims 1, 7, and 11, wherein the formulation comprises 5-15% dry matter of microalgae and/or is diluted to 0.3-0.5% v/v in water prior to application.

23. The method of any one of claims 1, 7, and 11, wherein the formulation is a liquid, and wherein the formulation is applied at a rate of 0.5-20 L/ha, optionally 1-10 L/ha.

24. The method of any one of claims 1, 7, and 11, wherein the formulation is a granule formulation, and wherein the formulation is applied at a rate of 1-20 kg/ha, optionally 5-15 kg/ha.

25. A method for improving host plant resistance to abiotic stress, the method comprising the step of:
a) applying a microalgae-based formulation to the host plant.

26. A method for improving host plant recovery from abiotic stress, the method comprising the step of:
a) applying a microalgae-based formulation to the host plant.

27. The method of claim 25 or 26, wherein the abiotic stress is water stress, temperature stress, sun stress, salinity stress, wind stress, herbicidal stress, or heavy metal stress.

28. The method of claim 25 or 26, wherein the abiotic stress is drought, heat, cold, excess salinity, herbicide exposure, strong winds, heavy metals, flooding, or excessive sunlight.

29. The method of claim 25 or 26, wherein the abiotic stress is water stress.

30. The method of claim 25 or 26, wherein the abiotic stress is drought.

31. The method of claim 25 or 26, wherein the abiotic stress is temperature stress.

32. The method of claim 25 or 26, wherein the abiotic stress is herbicidal stress.

33. The method of claim 25 or 26, wherein the abiotic stress is salinity stress.

34. A method for improving a host plant's resistance to and/or recovery from abiotic stress, the method comprising the step of:
a) applying a microalgae-based formulation to the host plant,
wherein the abiotic stress is drought.

35. The method of any one of claims 25-34, wherein the method decreases the damage to a growing parameter, production parameter, or biostimulant parameter of the host plant incurred by exposure to the abiotic stress.

36. The method of any one of claims 25-34, wherein the method decreases loss of yield as a result of exposure to the abiotic stress.

37. The method of any one of claims 25-34, wherein the method improves recovery from the damage to a growing parameter, production parameter, or biostimulant parameter of the host plant incurred by exposure to the abiotic stress.

38. The method of any one of claims 25-34, wherein the method decreases the amount of recovery time required by the host plant following exposure to the abiotic stress.

39. The method of any one of claims 25-34, wherein the method upregulates expression of genes involved in resistance to abiotic stress and/or drought resistance.

40. The method of any one of claims 25-34, wherein the method upregulates expression of a gene encoding an ABA receptor, a protein phosphatase 2C, an SNF1-related protein kinase 2, an ABRE-binding protein, an ABRE-binding factor, and/or a mitogen-activated protein kinase.

41. The method of any one of claims 25-34, wherein the method upregulates expression of a PYL, PP2C, SnRK2, AREB, ABF, and/or MAPK gene.

42. The method of any one of claims 25-34, wherein the formulation comprises multiple species of microalgae.

43. The method of any one of claims 25-34, wherein the formulation comprises microalgae from a phylum selected from the list consisting of: Chlorophyta, Cryptophyta, Cyanophyta, Euglenophyta, Heterokontophyta, or Rhodophyta.

44. The method of any one of claims 25-34, wherein the formulation comprises microalgae from a genus selected from the list consisting of: Chlorella, Scenedesmus, Nannochloropsis, Muriellopsis, Isochrysis, Tisochrysis, Desmodesmus, Haematococcus, Arthrospira, and Anabaena.

45. The method of any one of claims 25-34, wherein the formulation comprises whole-cell microalgae powder, optionally wherein the formulation comprises 0.1-50 g/L or 0.8-20 g/L of whole-cell microalgae powder.

46. The method of any one of claims 25-34, wherein the formulation comprises digested microalgae solution (“DMS”), optionally wherein the formulation comprises 0.3-0.5% v/v DMS and/or 5-15% dry matter of microalgae.

47. The method of any one of claims 25-34, wherein the formulation is a liquid, and wherein the formulation is applied at a rate of 0.5-20 L/ha, optionally 1-10 L/ha.

48. The method of any one of claims 25-34, wherein the formulation is a granule formulation, and wherein the formulation is applied at a rate of 1-20 kg/ha, optionally 5-15 kg/ha.
